(19) United States
(12) Patent Application Publication
(10) Pub. No.: US 2003/0232855 A1
(43) Pub. Date: Dec. 18, 2003

(54) Therapeutic agent for allergic disease

(75) Inventors: Hiroyuki Iwamura, Osaka (JP); Yoshifumi Ueda, Osaka (JP)

Correspondence Address: FOLEY AND LARDNER, SUITE 500, 3000 K STREET NW, WASHINGTON, DC, 20007, US

(73) Assignee: Japan Tobacco Inc.

(21) Appl. No.: 10/375,057

(22) Filed: Feb. 28, 2003

Related U.S. Application Data
(63) Continuation of application No. PCT/JP02/13806, filed on Dec. 27, 2002.

(30) Foreign Application Priority Data
Dec. 27, 2001 (JP) 396981/2001

(51) Int. Cl. A61K 31/4706; A61K 31/4709; C 07D 4 1/02; C07D 215/36

(52) U.S. Cl. 514/312; 546/157

(57) ABSTRACT

In accordance with the invention, a therapeutic agent of allergic disease is provided, which contains a cannabinoid receptor-regulating substance, particularly a regulating substance selectively acting on peripheral cell type cannabinoid receptor, specifically N-(benzo[1,3]dioxol-5-ylmethyl)-7-methoxy-2-oxo-8-pentyloxy-1,2-dihydroquinoline-3-carboxamide or a pharmaceutically acceptable salt thereof; and the therapeutic agent of the invention shows effects on intractable allergic disease, for example asthma and atopic dermatitis.

### BACKGROUND OF THE INVENTION

1. Field of the Invention

The present invention relates to a novel use of cannabinoid receptor-regulating substance. More specifically, the invention relates to a use of a regulating substance selectively acting on cannabinoid receptor, particularly peripheral cell type receptor (also referred to as CB2) as a therapeutic agent for allergic disease. Additionally, the invention relates to a new use of N-(benzo[1,3]dioxol-5-ylmethyl)-7-methoxy-2-oxo-8-pentyloxy-1,2-dihydroquinoline-3-carboxamide or a pharmaceutically acceptable salt thereof as a therapeutic agent for allergic disease.

2. Background Art

With respect to Indian hemp and cannabinoid Hemp has been used-as a drug for analgesic action, antipyretic action, hypnotic action and the like, traditionally from the ancient time. In Japan, the Japanese Pharmacopoeia describes hemp as Indian hemp from 1886 to 1951 for use as analgesics and anesthetic agent. In USA, meanwhile, the Pharmacopoeia describes the alcohol solution of hemp as a pharmaceutical agent for rheumatoid, asthma, tonsillitis and the like.

On the other hand, hemp or Δ9-tetrahydrocannabiol (THC) as the main ingredient thereof for the expression of its psychological actions has been known to induce abnormality in vision and hearing, abnormal cognition over time and space, suggestibility elevation, reduction of thinking potency and spontaneity and memory disorders, to thereby cause distinct change in psychological function. Other pharmacological actions are so diverse, including for example, motion ataxia, irritability elevation, body temperature decrease, breathing suppression, heart rate elevation, catalepsy-triggering action, blood pressure increase, vasodilating action, immunosuppressive action, and hydrodipsia. Currently, the use thereof is under regulation.

A series of psychedelic substances in hemp are collectively referred to as cannabinoid. To date, 60 types or more of cannabinoid, including THC have been found.

Various artificial ligands stronger than naturally occurring cannabinoid have been developed. The receptors thereof have also been screened. Consequently, in 1988, the existence of a cannabinoid receptor was shown as a membrane component of murine brain. In 1991, subsequently, human cDNA was cloned. Alternatively, a protein with 44% homology to the receptor was found in human promyelocytic leukemia cell HL60. Thereafter, it was confirmed that the protein was distributed in peripheral tissues such as spleen. In 1993, Munro et al. proposed to call the brain receptor “CB1” and the receptor found in peripheral tissues “CB2”. Currently, the designated terms are generally used.

It is shown with respect to the biological distribution of CB1 that CB1 is observed in many tissues such as human testicle, human prostate, ovary, uterus, bone marrow, thymus, tonsil, pituitary gland, adrenal gland, heart, lung, stomach, large intestine, bile duct, and leukocyte, other than brain. However, the levels thereof are far lower than the level thereof in brain. In contrast, CB2 never exists in brain in rat but was observed in the monocyte in the spleen marginal band. In human spleen, leukocyte, tonsil, thymus and pancreas, CB2 exists at a far higher level than that of CB1.

It was also confirmed that the two subtypes (CB1 and CB2) of the receptor were substantially characterized and that for the receptor, endogenous ligands such as anandamide, and 2-arachidonoylglycerol existed. Then, the physiological roles thereof have been examined. Consequently, various findings have been under way of collection, such that CB2 suppresses the proliferation of T cell and B cell to induce apoptosis and exert immunosuppressive action, that CB1-defective knockout mouse never exerts the central action as observed via cannabinoid dosing, and that CB2-defective knockout mouse never suppresses the activation of helper T cell with cannabinoid.

Currently, difference in distribution and function between CB1 and CB2 has been examined on the basis of these findings, to make an attempt to apply agonists, antagonists, or inverse agonists individually specific to these subtypes to pharmaceutical products. In relation with CB1, for example, Parkinson's disease, Alzheimer's disease, memory disorders, senile dementia, multiple sclerosis, appetite loss, and pain have been targeted as subjects for creating pharmaceutical agents therefor. In relation with CB2, for example, immune disease, rheumatoid arthritis and inflammation have been targeted as subjects for creating pharmaceutical agents therefor. Among them, pharmaceutical agents selectively exerting actions on CB2, namely regulating substances selective for the cannabinoid receptor of peripheral cell type (also referred to as peripheral type or periphery) are expected to be safe with no central action. Because cannabinoid at a very low concentration has a central action in CB1, further, a CB2-selective regulating substance with lower CB1 action is desirable.

Currently, it is additionally known that non-selective cannabinoid receptor ligands include for example Δ9-THC, CP55940, WIN55212-2, HU-243, and HU-210, while CB1—selective ligands include for example SR141716A, LY320135, arachidonoyl-2′-chloroethylamide, and CP56667 and that CB2-selective ligands include for example SR144528, AM630, HU-308, JWH-051, and L-768242 (see for example non-patent reference 1 and non-patent reference 2).

With Respect to Allergy

Herein, allergic disease particularly including allergic dermatitis and allergic asthma is described.

Allergy is recognized as a hypersensitive biological reaction based on antigen-antibody reaction, so allergy is different from general inflammatory reaction involving characteristic accumulation of monocytes, macrophages, and neutrophils. Eosinophils, basophils and mast cells are largely involved in allergic reaction.

Allergic reaction is now classified in four types, generally, but these four reactions never occur independently in the body. Reactions of several types may simultaneously occur in some cases.

When antigen (allergen) invades in the body, first, the antigen is incorporated into antigen presenting cells such as macrophages. The antigen presenting cells transmit the information of the incorporated antigen to T cells. Further, the T cells order B cells to prepare antigen-specific IgE antibody. The IgE antibody is bound to mast cells, so that the mast cells fall into a sensitized state.

When the antigen again invades in the body so that the IgE antibody on the mast cells is bound to the antigen, various chemical mediator such as histamine, eosinophil chemotactic factor and leukotriene, and cytokines such as interleukin are released from the mast cells.

When such chemical mediator acts on bronchi, the smooth muscle of the bronchi constricts to cause mucosal swelling and sputum secretion to narrow its airway, leading to the occurrence of asthma attack. When such chemical mediator exerts its action on skin, inflammation, swelling and itching occur to cause dermal diseases such as urticaria. When such chemical mediator exerts its action on nasal mucus, vascular permeability is increased, so that exudate is drawn out of blood to cause swelling of nasal mucus and nasal occlusion or cause allergic rhinitis involving hiccup and a large volume of pituita via nervous irritation. When the reaction occurs in digestive tract, intestinal smooth muscle constricts to abnormally increase intestinal motion (vermiculation), causing digestive allergies such as abdominal pain, vomiting and diarrhea.

Because the reaction occurs within 30 minutes after the antigen invasion, the reaction is referred to as early phase allergic response or Type I allergic reaction. Generally, early phase allergic response disappears in about one hour. Typical diseases include for example anaphylaxis, allergic rhinitis, pollenosis, urticaria, and allergic gastrointestinal diseases.

Several hours to several days later, however, eosinophil with highly toxic chemical mediators are attracted toward eosinophils chemotactic factor and cytokines released from mast cell, to accumulate at sites with allergic reactions, so that the eosinophils release the chemical mediators, triggering tissue damages. This is referred to as “late phase allergic response”. When the reaction occurs in bronchi, epithelium of mucus is detached so that antigen can more readily invade in the bronchi, leading to the prolongation of the allergic reaction and the elevation of the hypersensitivity of airway, causing asthma to be intractable. This is referred to as late phase type asthmatic reaction. For example, such late phase reaction mainly occurs 4 to 8 hours later in case of asthma and mainly occurs 12 to 48 hours later in case of atopic dermatitis.

Type II allergic reaction is also referred to as cytolysis type, where complement exerts an action on IgM or IgG antibody bound to antigen, to open holes through cell membrane to lyse the cell. Alternatively, a reaction occurs, in which macrophages or killers cell act on the cells bound with the antibody to release damaging substances to damage the cell or tissue. Typical diseases thereof include for example hemolytic anemia, idiopathic thrombocytopenic purpura, myasthenia gravis, and Goodpasture syndrome.

In Type III allergic reaction, an antigen-antibody complex of an antigen and an antibody (IgG antibody) bound together cannot be sufficiently treated by phagocytes but is deposited on a tissue, where complement, macrophages and neutrophils accumulate to cause inflammation and damage the tissue. Typical diseases include for example acute glomerulonephritis induced by hemolytic streptococcus, rheumatoid arthritis, collagen disease, serum sickness, viral hepatitis, and allergic alveolitis.

Type IV allergic reaction is different from the Type I to III reactions in that no antibody is involved in Type IV allergic reaction. When antigen again infiltrates at a state of established sensitization, T cells release cytokines and migrates immune cells such as lymphocytes, neutrophils and macrophages to destroy the antigen and simultaneously induce inflammation, causing tissue damages. When the infiltrating antigen is a cell, killer T cell damages the cell. The reaction is generally completed in one to 2 days, which is also referred to as “delayed-type allergic reaction”. Type IV allergic reaction includes for example tuberculin reaction, tuberculosis lesion, post-organ grafting rejections, and dermatitis such as rash against Japanese lacquer (urushi) and rash against cosmetics.

Most of acute symptoms of common allergic disease such as allergic asthma, atopic dermatitis, allergic rhinitis and allergic conjunctivitis have been classified as early phase response. In recent years, however, it has been recognized that allergic asthma is not a transient immediate type hypersensitivity but is essentially chronic inflammation.

It has been known that asthma is classified as “allergic asthma” induced by allergen and non-allergic asthma induced not by specific allergen but by coldness, athletic motion and the like.

“Asthma” namely “bronchial asthma” has been characterized by its reversible airstream restriction (airway occlusion) and airway hypersensitivity. However, it is now elucidated that airway suffering from asthma involves the occurrence of chronic inflammation with characteristic features of detachment of airway epithelium, fibrosis of airway just below the basement membrane (hypertrophy of the basement membrane), and eosinophil accumulation. Currently, asthma is therefore recognized as chronic inflammatory disease. It is suggested that many inflammatory cells such as eosinophils, T cells and mast cells are involved in airway inflammation. It is considered that mast cells involvement in early phase response, eosinophil involvement in late phase response and the involvement of eosinophils and CD4-positive helper T cells in delayed type reaction are significant.

As to anti-asthma agent, therapeutic treatment of reversible airway occlusion mainly with bronchial dilator has been replaced with therapeutic treatment of chronic inflammation mainly with anti-inflammatory agent. For the therapeutic treatment of asthma attack, short-term acting β2 stimulator, short-term acting theophylline, anti-choline agent for inhalation, steroidal agents for injections or oral dosing are used, in a manner dependent on the symptom. For long-term control, further, steroidal agents for inhalation and oral dosing, sustained-release type theophylline, and long-term acting β2 stimulator as well as anti-allergic agents (mediator release-inhibitor, histamine H1 antagonists, leukotriene antagonists, thromboxane A2 inhibitors and antagonists, and Th2 cytokine inhibitors) are used. However, it is known that these agents have side effects such as suppression of adrenal function as observed in the steroidal agents. Some symptom (resistance) is also known, for which the effects of steroid and leukotriene antagonists are low. Therefore, additional anti-asthmatic agents are expected.

Atopic asthma or atopic dermatitis are symptoms of allergic disease with family history or anamnesis. Asthma and dermatitis of atopic type are frequently observed in children. Therefore, a therapeutic agent with less side effects in particular is desired.

In definition, “‘atopic dermatitis’ is a disease with a main lesion of eczema with itching, which repeats exacerbation and remission. Many of the patients have atopic predisposition.

Atopic Predisposition is Defined

by (1) family history, anamnesis (any disease or plural diseases of bronchial asthma, allergic rhinitis, conjunctivitis, and atopic dermatitis), or

as (2) predisposition readily generating IgE antibody”. Atopic dermatitis is discriminated from other inflammatory dermatitis.

Symptoms of atopic dermatitis include hypersensitivity and dryness of skin. Characteristic exanthema (erythematosus, papule, incrustation, lichen lesion, prurigo and the like) progress in chronic and recurrent course. Further, the symptoms induce complications such as Kaposi's sarcoma varicelliform eruption, viral infections (infection with herpes simplex virus and the like), impetigo, and infectious molluscum (cataract, retinodialysis, etc.)

It is also considered that early phase and late phase allergic response via IgE and mast cell and additionally delayed type allergic reaction via Langerhans cells and T cells are involved in the lesion of atopic dermatitis.

For the therapeutic treatment, pharmaceutical therapy is used, depending on the symptom, together with the removal of causes or exacerbating factors such as foods or mite, and with skincare (keeping skin clean and using moisturizing agents so as to prevent skin dryness).

For itching, anti-histamine agents are used. However the effect is not so distinct as in the case of urticaria.

For inflammation, principally, external steroidal application agents are used. Oral dosage forms of anti-histamine agents or anti-allergy agents are used in a supplementary manner. However, it is said that it is difficult to control dermatitis with them alone. Generally, atopic dermatitis is hard to cure and many individuals reject steroidal agents due to the side effects. Thus, a new pharmaceutical agent is demanded. In recent years, an immunosuppressor tacrolimus ointment has been used and shows some effect. The side effects thereof draw concerns alike, so the use is restricted. For therapeutic treatment of symptoms with severe damages on skin lesions and with difficulty in external application, symptoms occurring at sensitive sites with originally thin epithelium such as face and mucus, and diseases in the inner layer of epithelium or over a wide area of body, additionally, the development of an oral agent with ready handling and a safety profile is desired.

JP-A-2000-256323 (WO 00/40562) as an application filed by the present applicant describes the 2-oxoquinoline compound represented by the following general formula as a cannabinoid receptor-regulating substance.

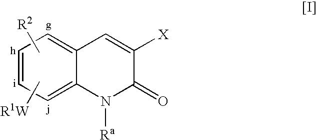

(In the formula, the symbols are as described therein.)

Additionally, JP-A-2000-256323 describes N-(benzo[1,3]dioxol-5-ylmethyl)-7-methoxy-2-oxo-8-pentyloxy-1,2-dihydroquinoline-3-carboxamide (referred to as Compound A hereinafter) as an example of the 2-oxoquinoline compound.

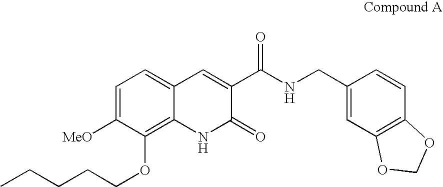

Further, the gazette thereof describes concerning the application of the cannabinoid receptor-regulating substance that “due to the finding of a receptor of peripheral cell type, for example such receptor on macrophages (see non-patent reference 6), an agonist of the receptor of peripheral cell type is under way of development, which has anti-inflammatory action and anti-allergy action via the regulation of immune reaction and which originally has immunoregulatory action” and that “the development of a peripheral cell type receptor-selective regulating substance is particularly desired because a pharmaceutical agent with an action selective for peripheral cell type cannabinoid receptor never exerts central actions such as hypothermia and catalepsy as the side effects but can have a safety profile”, and that “such regulating substance is useful as cannabinoid receptor (particularly peripheral type cannabinoid receptor)—regulating substance, immunomodulator, therapeutic agent of autoimmune disease, anti-inflammatory agent and anti-allergic agent”.

Additionally, the gazette thereof describes a test of selective binding to peripheral cell type cannabinoid receptor (CB2), a carrageenan-induced paw edema model test, and a suppressive test of inflammation and bleeding of pancreas in rat taurocol pancreatitis model (see patent reference 1).

Although the gazette includes specific disclosure about the anti-inflammatory action, no effect on allergic disease is demonstrated. It is needless to say that the gazette neither describes any effect on atopic dermatitis and allergic asthma.

Meanwhile, a related-art reference describes that the Compound A and SR144528 described below are CB2-selective ligands and that they function as CB2 inverse agonists. In more detail, the related-art reference describes that the Compound A and SR144528 below elevate the generation of cyclic adenosine-phosphate (cAMP) on stimulation with forskolin as an activating agent of adenylate cyclase on the CB2 expressing CHO cells, in other words that the Compound A and SR144528 function as CB2 inverse agonists. The reference concurrently describes about a general finding that THC reduces cAMP generation at the test (see non-patent reference 3).

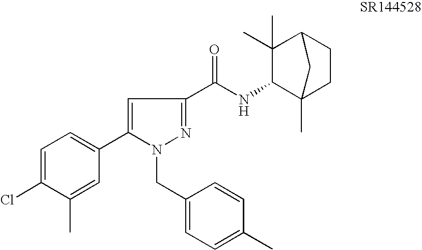

Some known patent publications describe about the anti-allergic effect of cannabinoid regulating substance.

JP-A-52-113976 (U.S. Pat. No. 4,179,517) describes the effect of a THC derivative on the prevention of asthma attack and describes asthma, allergy and the like as the indication (see patent reference 2).

JP-T-2002-511411 (WO 99/52524) describes that cannabinoid such as cannabidiol can be used for the therapeutic treatment of inflammatory diseases such as asthma but also cites a reference telling that cannabidiol never binds to CB1 or CB2 (see patent reference 3).

WO 01/64212 describes that cannabinoid regulating substance, preferably CB1 agonist can be used for the therapeutic treatment of muscle diseases, for example asthma and bronchitis (see patent reference 4).

WO 01/95899 describes the anti-inflammatory action of cannabidiol derivative on arachidonate-induced ear edema (see patent reference 5).

WO 01/89589 describes a method for ameliorating cough, including local administration of cannabinoid to regulate CB1 receptor existing in peripheral cells (see patent reference 6).

WO 00/16756 discloses a cannabinoid regulating substance and dermal diseases (atopic dermatitis, and the like), respiratory diseases (asthma and the like), allergic rhinitis and the like as the indication. WO 00/16756 also describes that the compound is nonetheless CB1-selective and regulates CB1 receptor existing in peripheral cells (see patent reference 7).

JP-T-8-504195 (WO 94/12466) describes that a ligand for cannabinoid receptor exerts anti-inflammatory, anti-asthmatic activities and the like (see patent reference 8).

JP-A-6-73014 (U.S. Pat. No. 5,624,941) and JP-A-7-324076 (U.S. Pat. No. 5,462,960) describe that ligands for cannabinoid receptor can be used for the therapeutic treatment of thymus disorders, asthma, immunoregulation and the like (see patent references 9 and 10).

WO 01/98289 describes that compounds of Δ6-tetrahydrocannabiol type can be used for the therapeutic treatment of inflammation, lung diseases such as asthma and chronic occlusive lung disease, autoimmune disease and the like and that the action is however via inhibition of prostaglandin synthesis, inhibition of tumor necrosis factor generation, cyclooxygenase inhibition, and inhibition of nitric oxide generation, in addition to blocking of N-methyl-D-aspartic acid receptor and anti-oxidative activity (see patent reference 11).

WO 02/26702 describes that cannabinoid receptor-regulating substances, particularly agonists are effective for asthma, allergy, dermal diseases and the like (see patent reference 12).

WO 01/87297 describes that CB1 regulating substance can be used for the therapeutic treatment of dermal necrosis such as psoriasis (see patent reference 13).

WO 02/42248 describes that cannabinoid receptor-binding agents, particularly CB1 agonist can be used for asthma, rhinitis, and inflammatory dermal diseases (see patent reference 14).

WO 02/47691 describes that cannabinoid receptor agonist can be used for the therapeutic treatment of inflammation and the like (see patent reference 15).

However, these publications never disclose any data verifying that the compounds show therapeutic effects on allergic disease or never describe that the compounds selectively act on CB2 or include any description suggesting the action.

Further, some of the publications include descriptions about the pharmacological action of cannabinoid regulating substance selective to CB2.

JP-T-11-500411 (WO 96/18391) describes that CB2 regulating substance can be used for the therapeutic treatment of immune system disorders, chronic respiratory disorders (asthma and the like) and the like, and further describes that CB2 expression in mast cells and non-immune cells (for example, cerebellar granule, cerebellum, and heart) (see patent reference 16) was observed.

JP-T-11-501615 (WO 96/18600) describes that CB2 regulating substances can be used for the therapeutic treatment of autoimmune disease, chronic inflammation, respiratory disorders (asthma and the like) and the like (see patent reference 17).

JP-T-10-508870 (WO 96/25397) describes that CB2 regulating substance can be used for the therapeutic treatment of lung disorders (asthma, chronic bronchitis and the like), allergic reactions (rhinitis, contact dermatitis, conjunctivitis, and the like) and immune system disorders (see patent reference 18).

JP-T-11-507937 (U.S. Pat. No. 6,013,648) describes an agent acting on CB2 and describes autoimmune disease, infectious disease and allergic disease (specifically, acute hypersensitivity, and asthma) as the indication thereof. However, the agent acting on CB2 has selectivity on CB2 but suppresses cAMP generation on forskolin stimulation (see patent reference 19).

JP-T-2000-502080 (U.S. Pat. No. 5,925,768) describes compounds with affinity for CB2 receptor and describes immune diseases for example allergic disease (immediate type hypersensitivity or asthma) as the indication. The reference however describes that the compounds are CB2 receptor antagonists (see patent reference 20).

JP-T-2001-508799 (WO 98/31227) describes that CB2 regulating substances, particularly antagonists can be used for the therapeutic treatment of immune diseases, inflammation and the like (see patent reference 21).

JP-T-2001-516361 (WO 98/41519) describes that CB2 regulating substances, particularly agonists can be used for the therapeutic treatment of immune diseases, inflammation and the like (see patent reference 22).

JP-T-2001-515470 (U.S. Pat. No. 6,262,112) describes that cannabinoid, particularly CB1 agonist are effective for the therapeutic treatment of allergic disease, asthma, inflammatory dermal diseases and/or dermal diseases ascribed to immunological causes. Additionally, JP-T-2001-515470 describes that some of the compounds are effective for CB2 (see patent reference 23).

WO 99/57107 describes that CB2-selective regulating substances can be used for anti-inflammation and immunoregulation (see patent reference 24).

JP-T-2002-523395 (WO 00/10967) and JP-T-2002-523396 (WO 00/10968) describe that CB1 agonist and CB2 agonist can be used individually for the therapeutic treatment of dermal diseases and the like (see patent references 25 and 26).

JP-T-2002-539246 (WO 00/56303) describes that CB2-selective agonists can be used for the therapeutic treatment of immune diseases (see patent reference 27).

WO 01/4083 describes that CB2-selective regulating substances, particularly agonists can be used for the therapeutic treatment of inflammation, immunological diseases, such as atopic dermatitis, allergic dermatitis, and asthma. The publication however describes that the compounds suppress cAMP-increase (see patent reference 28).

WO 01/19807 describes that CB2-selective regulating substances, particularly agonists have anti-inflammatory and immunosuppressive actions and describes the test results of experiments in sheep erythrocyte-induced delayed type hypersensitivity model. The publication however describes that the compounds suppress cAMP-increase (see patent reference 29).

WO 01/29007 describes that cannabinoid regulating substances are used for anti-inflammation and the regulation of immune system and describes that some of the compounds are antagonists, while the remaining compounds are agonists and additionally describes CB2-selective regulating substances based on the binding assay results (see patent reference 30).

WO 01/28497 describes that CB2-selective regulating substances, particularly agonists have anti-inflammatory actions and the like (see patent reference 31).

WO 01/32169 describes that CB2-selective agonists are used for anti-inflammation and the therapeutic treatment of autoimmune disease and the like (see patent reference 32).

WO 01/28329 describes that CB2-selective substances are used for anti-inflammation and the therapeutic treatment of autoimmune disease and the like (see patent reference 33).

WO 01/28557 describes that cannabinoid receptor-regulating substances are used for anti-inflammation and the therapeutic treatment of autoimmune disease and the like and discloses test data showing that some of the compounds are CB2-selective regulating substances (see patent reference 34).

WO 01/32629 describes that CB2 antagonists are used for anti-inflammation and the therapeutic treatment of immune diseases and the like (see patent reference 35).

WO 01/58869 describes that CB agonists, particularly CB2 agonists are used for the therapeutic treatment of respiratory diseases, particularly asthma and bronchitis. Further, the publication describes that the agonists suppress mucin generation from lung epithelial cells (see patent reference 36).

WO 01/96330 discloses CB2-binding compounds and respiratory diseases, for example asthma and bronchitis, and inflammatory diseases as the indication (see patent reference 37).

WO 02/10135 describes that CB2 agonists are effective for the therapeutic treatment of asthma, nasal allergy, atopic dermatitis, autoimmune disease and the like. Further, the publication shows test results indicating that the compounds suppress cAMP generation (see patent reference 38).

WO 02/42269 describes that CB2 agonists are effective for the therapeutic treatment of immune diseases such as psoriasis, allergic disease such as hypersensitivity, asthma, allergic rhinitis, and contact dermatitis, inflammatory diseases such as arthritis, and the like (see patent reference 39).

WO 02/58636 describes that cannabinoid-like compounds, particularly CB2-selective compounds are used for anti-inflammation, the regulation of immune system and the like. Further, the publication describes that the compounds are agonists suppressing cAMP generation (see patent reference 40).

WO 02/60447 describes CB1-selective regulating substances and CB2-selective regulating substances. Further, the publication describes that the CB2-selective regulating substances, particularly antagonists are used for anti-inflammation, the regulation of immune system and the like (see patent reference 41).

WO 02/53543 describes that compounds with CB2 affinity are used as anti-inflammatory agents, immunosuppressors and the like. The publication also describes that some compounds exert agonist actions as assayed by the measurement of the amount of cAMP generated on forskolin stimulation and also describes a test method using sheep erythrocyte-induced delayed type hypersensitivity model. (see patent reference 42).

WO 02/72562 describes that compounds with CB2 affinity, particularly agonists, are used as anti-inflammatory agents and immunosuppressors and the like. The publication also describes that some compounds exert agonist actions as assayed by the measurement of the amount of cAMP generated on forskolin stimulation and also describes a test method using sheep erythrocyte-induced delayed type hypersensitivity model (see patent reference 43).

WO 02/62750 describes that cannabinoid regulating substances, particularly compounds binding to CB2 are effective for the therapeutic treatment of atopic dermatitis, allergy, asthma, chronic occlusive lung diseases, bronchitis, and the like (see patent reference 44).

WO 02/85866 describes that CB2-selective agonists are effective for pain treatment (see patent reference 45).

These publications never disclose any data verifying that the compounds show therapeutic effects on allergic disease or any data showing that the compounds are effective for allergic dermatitis, atopic dermatitis, allergic asthma, early phase asthma response, late phase asthma response, and airway hypersensitivity. Furthermore, the publications never show that the compounds show their therapeutic effects via the action of CB2 inverse agonists or never include any description suggesting the action.

Further, the publications or the references never describe any definite grounds or verified facts that cannabinoid receptor-regulating substances, particularly CB2-selective regulating substances, particularly CB2-selective inverse agonists are effective for allergic disease. Further, the publications or the references never include any description showing that these regulating substances are effective for allergic dermatitis, atopic dermatitis, allergic asthma, early phase asthma response, late phase asthma response and airway hypersensitivity.

As described above, findings about the relation between the action on cannabinoid receptors and pathogenesis are so diverse. For clinical application of CB2-selective regulating substances, in particular, any definite finding as to whether CB2-selective regulating substances should be agonists or antagonists or inverse agonists has not yet been obtained.

In such circumstances, cannabinoid regulating substances for use as anti-allergy agents have not yet been developed.

For the evaluation of the pharmacological actions in accordance with the invention, additionally, the present inventors used as experimental model animals effective for the judgment of anti-allergy effect, DNFB-induced allergic dermatitis mouse with induced inflammation similar to atopic dermatitis (see non-patent reference 4), IgE-dependent allergic dermatitis model mouse with induced triphase (early phase, late phase, very late phase) dermatitis (see non-patent reference 5). These experimental models are used as models appropriate for the evaluation of anti-allergic actions, particularly for the evaluation of pharmacological actions on atopic dermatitis.

[Patent Reference 1]

JP-A-2000-256323 (Examples 3 to 5, page 29; and page 6, right column, line 42 to page 7, left column, line 1; page 65, right column, line 43 to line 46; page 63, left column, line 16 to page 65, left column, line 37)

[Patent Reference 2]

JP-A-52-113976 (page 3, lower right column, line 1 to line 4; page 8, upper right column, line 12 to line 17)

[Patent Reference 3]

JP-T-2002-511411 (page 6, column No. 0005; page 7, column No. 0009)

[Patent Reference 4]

WO 01/64212 (page 4, line 1 to line 29)

[Patent Reference 5]

WO 01/95899 (page 20, line 7 to page 23, line 23)

[Patent Reference 6]

WO 01/89589 (page 2, line 15 to page 4, line 2; FIGS. 2B, 2C)

[Patent Reference 7]

WO 00/16756 (page 13, line 18 to page 15, line 14; table, page 30, line 13 to page 32; page 43, line 4 to page 44, line)

[Patent Reference 8]

JP-T-8-504195 (page 12, table II; page 16)

[Patent Reference 9]

JP-A-6-73014 (page 6, left column, line 28 to line 50)

[Patent Reference 10]

JP-A-7-324076 (page 8, left column, line 4 to line 34)

[Patent Reference 11]

WO 01/98289 (page 5, line 13 from the bottom to page 7, line 11; page 12, line 7 to line 13)

[Patent Reference 12]

WO 02/26702

[Patent Reference 13]

WO 01/87297 (page 3, line 9 to line 15; page 10, line 7 to line 13)

[Patent Reference 14]

WO 02/42248 (page 6, line 5 from the bottom to page 7, line 20, page 12, line 14 to line 17)

[Patent Reference 15]

WO 02/47691 (page 2, column No. 0006; page 3, line 4 to the last line)

[Patent Reference 16]

JP-T-11-500411 (page 9, line 12 to page 11, line 12; page 65, line 22 to page 67, line 6)

[Patent Reference 17]

JP-T-11-501615 (page 16, line 16 to line 21; page 52, line 14 to page 54, line 7)

[Patent Reference 18]

JP-T-10-508870 (page 13, line 11 to line 12; page 34, line 7 to line 22)

[Patent Reference 19]

JP-T-11-507937 (page 13, line 10 to line 22; page 66, line 14 to page 67, line 5)

[Patent Reference 20]

JP-T-2000-502080 (page 42, line 19 to page 44, line 2)

[Patent Reference 21]

JP-T-2001-508799 (page 14, line 5 to line 14; page 27, line 9 to line 18)

[Patent Reference 22]

JP-T-2001-516361 (page 6, line 18 to page 7, line 2; page 14, line 17 to line 18)

[Patent Reference 23]

JP-T-2001-515470 (page 86, line 7 to page 87, line 14)

[Patent Reference 24]

WO 99/57107 (page 1, line 1 to page 2, line 13; table, page 22)

[Patent Reference 25]

JP-T-2002-523395 (page 65, line 9 to page 66, line 20)

[Patent Reference 26]

JP-T-2002-523396 (page 78, line 3 from the bottom to page 80, line 8)

[Patent Reference 27]

JP-T-2002-539246 (page 53, line 5 to page 54, line 23; page 64, line 9 from the bottom to page 65, line 5)

[Patent Reference 28]

WO 01/4083 (page 50, line 9 to page 56, line 12)

[Patent Reference 29]

WO 01/19807 (page 27, line 11 to page 28, line 8; page 134, line 7 from the bottom to page 138, the last line)

[Patent Reference 30]

WO 01/29007 (page 4, line 6 to line 25; table 1, page 8)

[Patent Reference 31]

WO 01/28497 (page 1, line 4 from the bottom to page 3, line 6; page 9, line 21 to line 26)

[Patent Reference 32]

WO 01/32169 (page 3, line 18 to page 4, the last line)

[Patent Reference 33]

WO 01/28329 (page 2, line 1 to page 3, line 14)

[Patent Reference 34]

WO 01/28557 (page 2, line 5 to page 5, line 15; table, page 7)

[Patent Reference 35]

WO 01/32629

[Patent Reference 36]

WO 01/58869 (page 2, line 1 to line 8; page 44, line 4 from the bottom to page 46, line 15)

[Patent Reference 37]

WO 01/96330 (page 7, line 27 to page 8, line 9; page 56, line 9 to line 29)

[Patent Reference 38]

WO 02/10135 (page 71, line 10 to page 72, line 11)

[Patent Reference 39]

WO 02/42269

[Patent Reference 40]

WO 02/58636 (page 7, line 5 to page 8, line 25; page 29, line 18 to line 25)

[Patent Reference 41]

WO 02/60447 (page 6, line 1 to page 7, line 2; page 8, line 7 to line 17; table, page 9)

[Patent Reference 42]

WO 02/53543 (page 85, line 4 to the last line; page 278, line 4 to page 281, line 15)

[Patent Reference 43]

WO 02/72562 (page 29, line 22 to page 30, line 18; page 120, line 5 to page 123, line 19)

[Patent Reference 44]

WO 02/62750 (page 3, line 14 to page 4, the last line)

[Patent Reference 45]

WO 02/85866 (page 1, line 4 to line 8; page 8, line 31 to page 9, line 3)

[Non-Patent Reference 1]

Biology and Chemistry, Yamamoto Shozo, et al., ed., Vol. 39, No. 5, pp. 293-300, 2001

[Non-Patent Reference 2]

Expert Opinion on Therapeutic Patents, Vol. 12, No. 10, 1475-1489, 2002

[Non-Patent Reference 3]

The Journal of Pharmacology and Experimental Therapeutics, Vol. 296, No. 2, pp. 420-425 (table 1 on page 422; table 3 on page 423)

[Non-Patent Reference 4]

Journal of Allergy Clinical Immunology, Vol. 100, No. 6, Part 2, pp. 39-44, December 1997

[Non-Patent Reference 5]

Pharmacology, Vol. 60, No. 2, pp. 97-104, February 2000

[Non-Patent Reference 6]

Munro et al., Nature, Vol. 365, pp. 61-65, 1993

As described above, cannabinoid receptor-regulating substances have not yet been applied successfully as pharmacological product. Therefore, the effective use thereof has been examined.

### SUMMARY OF THE INVENTION

Thus, it is an object of the invention to provide a cannabinoid receptor-regulating substance, particularly a novel therapeutic agent of allergic disease, which contains as the active ingredient a regulating substance selective for the peripheral cell type cannabinoid receptor.

So as to attain the object, the inventors have made investigations. Consequently, the inventors have verified that a selective CB2-regulating substance very effectively works for allergic disease such as allergic asthma, atopic dermatitis, allergic rhinitis, and allergic conjunctivitis. Thus, the invention has been achieved. The pharmacological product of the invention is effective as a therapeutic agent of allergic asthma and atopic dermatitis, in particular. This fact, namely the effect of the invention is far superior to the effect indicated by the JP-A-2000-256323 (WO 00/40562), surprisingly for the inventors themselves.

In more detail, the invention is described in the following aspects.

1. A therapeutic agent of allergic disease, which contains as the active ingredient a cannabinoid receptor-regulating substance.

2. A therapeutic agent of allergic disease as described in the first aspect, where the cannabinoid receptor-regulating substance is a regulating substance selective for the peripheral cell type cannabinoid receptor.

3. A therapeutic agent of allergic disease as described in the first or second aspect, where the cannabinoid receptor-regulating substance is a 2-oxoquinoline compound represented by the following general formula [I] or a pharmaceutically acceptable salt thereof:

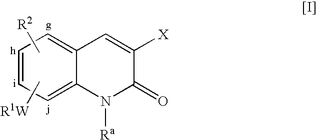

[in the formula,

W represents —O—, —S(O)ₜ—, —CR³R⁴—, —NR⁵—, —NR⁵CO—, —CONR⁵—, —COO— or —OCO— (in the formulas, R³ and R⁴ may be the same or different and independently represent hydrogen atom or alkyl; R⁵ represents hydrogen atom or alkyl; and t represents 0 or an integer of 1 and 2);

R¹ represents hydrogen atom, alkyl, alkenyl, alkynyl, aryl, arylalkyl, heteroaryl, heteroarylalkyl, cycloalkyl or cycloalkylalkyl; the individual groups in R¹ except for hydrogen atom may or may not be substituted with alkylamino, amino, hydroxyl group, alkoxy, carboxyl, alkoxycarbonyl, acyl, acyloxy, acylthio, mercapto, alkylthio, alkylsulfinyl or alkylsulfonyl; the individual groups except for hydrogen atom and alkyl may or may not be substituted with alkyl;

R² represents hydrogen atom, alkyl, —OR⁶ (in the formula, R⁶ represents hydrogen atom, alkyl, alkenyl, alkynyl, aryl, arylalkyl, heteroaryl, heteroarylalkyl, cycloalkyl or cycloalkylalkyl), —NR⁷R⁸ (in the formula, R⁷ and R⁸ may be the same or different and individually represent hydrogen atom, alkyl, alkenyl, alkynyl, acyl, aryl, arylalkyl, heteroaryl, heteroarylalkyl, cycloalkyl or cycloalkylalkyl; otherwise, R⁷ and R⁸ together with the adjacent nitrogen atom may form heteroaryl), or —(CH₂)_{u′}—S(O)ᵤR⁹ (in the formula, R⁹ represents hydrogen atom, alkyl, alkenyl or alkynyl; u and u′ independently represent 0 or an integer of 1 and 2),

where the individual groups in R² except for hydrogen atom may or may not be substituted with alkylamino, amino, hydroxyl group, alkoxy, alkoxycarbonyl, acyl, acyloxy, acylthio, mercapto, alkylthio, alkylsulfinyl or alkylsulfonyl; the individual groups except for hydrogen atom and alkyl may or may not be substituted with alkyl;

R^{a} represents hydrogen atom or alkyl;

X represents —COOR^{b}, —CONH₂, —CONR^{c}-(Alk^{a})ᵣ-R, —(CH₂)ₚ—OC(═Y)—NR^{d}—(Alk^{b})ₛ—R, —(CH₂)_{q}—NR^{e}—C(═Z)—(NR^{f})_{w}—(Alk^{c})ᵥ—R, —(CH₂)ₚ—OH or —(CH₂)_{q}—NR^{e}R^{e′}

{in the formula, R^{b}, R^{c}, R^{d} and R^{f} independently represent hydrogen atom or alkyl; R^{e} and R^{e′} independently represent hydrogen atom or alkyl; or R^{e} and R^{e′} together with the adjacent nitrogen atom may form heteroaryl;

Alk^{a}, Alk^{b} and Alk^{c} independently represent alkylene or alkenylene; the alkylene and alkenylene individualy may or may not be substituted with hydroxyl group, carboxyl, alkoxycarbonyl, alkyl (the alkyl may or may not be substituted with hydroxyl group, alkoxy or alkylthio) or —CONR¹⁰R¹¹ (in the formula, R¹⁰ and R¹¹ may be the same or different and independently represent hydrogen atom or alkyl; or R¹⁰ and R¹¹ together with the adjacent nitrogen atom may form heteroaryl);

R represents aryl, heteroaryl, cycloalkyl, benzene-fused cycloalkyl or

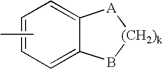

(in the formula, A and B independently represent oxygen atom, nitrogen atom or sulfur atom; and k represents an integer of 1 to 3), where the aryl and the heteroaryl individually may or may not be substituted with alkyl which may or may not be substituted with hydroxyl group, hydroxyl group, alkoxy, alkenyloxy, acyl, acyloxy, halogen atom, nitro, amino, sulfonamide, alkylamino, aralkyloxy, pyridyl, piperizino, carboxyl, alkoxycarbonyl, acylamino, aminocarbonyl, cyano or glucuronate residue; the cycloalkyl may or may not be substituted with hydroxyl group, alkoxy or ═O; the benzene-fused cycloalkyl may or may not be substituted with hydroxyl group or alkoxyl;

r, s, v and w independently represent 0 or 1; Y and Z independently represent nitrogen atom, oxygen atom or sulfur atom; and p and q independently represent an integer of 1 to 4}].

4. A therapeutic agent of allergic disease as described in the third aspect, where W is —O—; R¹ is hydrogen atom or alkyl (the alkyl is as described above); R² is —OR⁶ (R⁶ is as described above); R is aryl, heteroaryl or

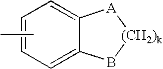

(where the aryl, the heteroaryl and the individual symbols in the formula are as described above).

5. A therapeutic agent of allergic disease as described in the first or second aspect, where the cannabinoid receptor-regulating substance is a 2-oxoquinoline compound represented by the following formula [I′] or a pharmaceutically acceptable salt thereof:

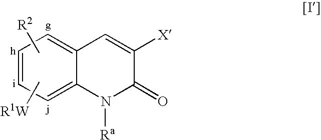

[in the formula, W represents —O—, —S(O)ₜ—, —CR³R⁴—, —NR⁵—, —NR⁵CO—, —CONR⁵—, —COO— or —OCO— (in the formula, R³ and R⁴ may be the same or different and individually represent hydrogen atom or alkyl; R⁵ represents hydrogen atom or alkyl; and t represents 0 or an integer of 1 and 2); R¹ represents hydrogen atom, alkyl, alkenyl, alkynyl, aryl, arylalkyl, heteroaryl, heteroarylalkyl, cycloalkyl or cycloalkylalkyl; the individual groups in R¹ except for hydrogen atom may or may not be substituted with alkylamino, amino, hydroxyl group, alkoxy, carboxyl, alkoxycarbonyl, acyl, acyloxy, acylthio, mercapto, alkylthio, alkylsulfinyl or alkylsulfonyl; the individual groups except for hydrogen atom and alkyl may or may not be substituted with alkyl;

R² represents hydrogen atom, alkyl, —OR⁶ (in the formula, R⁶ represents hydrogen atom, alkyl, alkenyl, alkynyl, aryl, arylalkyl, heteroaryl, heteroarylalkyl, cycloalkyl or cycloalkylalkyl), —NR⁷R⁸ (in the formula, R⁷ and R⁸ may be the same or different and individually represent hydrogen atom, alkyl, alkenyl, alkynyl, acyl, aryl, arylalkyl, heteroaryl, heteroarylalkyl, cycloalkyl or cycloalkylalkyl; otherwise, R⁷ and R⁸ together with the adjacent nitrogen atom may form heteroaryl), or —(CH₂)_{u′}—S(O)ᵤR⁹ (in the formula, R⁹ represents hydrogen atom, alkyl, alkenyl or alkynyl; u and u′ independently represent 0 or an integer of 1 and 2),

where the individual groups in R² except for hydrogen atom may or may not be substituted with alkylamino, amino, hydroxyl group, alkoxy, alkoxycarbonyl, acyl, acyloxy, acylthio, mercapto, alkylthio, alkylsulfinyl or alkylsulfonyl; the individual groups except for hydrogen atom and alkyl may or may not be substituted with alkyl;

R^{a} represents hydrogen atom or alkyl;

X′ represents —CONR^{c}-(Alk^{a})ᵣ-R, —(CH₂)ₚ—OC(═Y)—NR^{d}-(Alk^{b})ₛ-R or (CH₂)_{q}—NR^{e}—C(═Z)—(NR^{f})_{w}—(Alk^{c})ᵥ—R {in the formula, R^{c}, R^{d}, R^{e} and R^{f} independently represent hydrogen atom or alkyl; Alk^{a}, Alk^{b} and Alk^{c} independently represent alkylene or alkenylene; the alkylene and the alkenylene individually may or may not be substituted with hydroxyl group, carboxyl, alkoxycarbonyl, alkyl (the alkyl may or may not be substituted with hydroxyl group, alkoxy or alkylthio) or —CONR¹⁰R¹¹ (in the formula, R¹⁰ and R¹¹ may be the same or different and individually represent hydrogen atom or alkyl or R¹⁰ and R¹¹ together with the adjacent nitrogen atom may form heteroaryl; R represents aryl, hetero aryl, cycloalkyl, benzene-fused cycloalkyl or

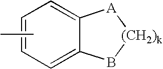

(in the formula, A and B independently represent oxygen atom, nitrogen atom or sulfur atom; and k represents an integer of 1 to 3),where the aryl and the heteroaryl individually may or may not be substituted with alkyl which may or may not be substituted with hydroxyl group, hydroxyl group, alkoxy, alkenyloxy, acyl, acyloxy, halogen atom, nitro, amino, sulfonamide, alkylamino, aralkyloxy, pyridyl, piperizino, carboxyl, alkoxycarbonyl, acylamino, aminocarbonyl, cyano or glucuronate residue; the cycloalkyl may or may not be substituted with hydroxyl group, alkoxy or ═O; the benzene-fused cycloalkyl may or may not be substituted with hydroxyl group or alkoxyl;

r, s, v and w independently represent 0 or 1; Y and Z independently represent nitrogen atom, oxygen atom or sulfur atom; and p and q independently represent an integer of 1 to 4}], under the provision (a) that 2-oxoquinoline is substituted at position j with WR¹ when R² is hydrogen atom and the provision (b) that 1,2-dihydro-6,7-dimethoxy-2-oxo-N-(phenylmethyl)-3-quinolinecarboxamide and N-(1,2-dihydro-6,7-dimethoxy-2-oxo-3-quinolyl)benzamide are excluded.

6. A therapeutic agent of allergic disease as described in the fifth aspect, where X′ is —CONR^{c}-(Alka)ᵣ-R.

7. A therapeutic agent of allergic disease as described in the fifth aspect, where X′ is —(CH₂)ₚ—OC(═Y)—NR^{d}-(Alk^{b})ₛ-R or (CH₂)_{q}—NR^{e}—C(═Z)—(NR^{f})_{w}—(Alk^{c})ᵥ—R.

8. A therapeutic agent of allergic disease as described in the fifth through seventh aspects, where R is aryl, or heteroaryl or

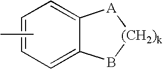

(where the aryl, the heteroaryl and individual symbols in the formula are as described above).

9. A therapeutic agent of allergic disease as described in the fifth through seventh aspects, where R is

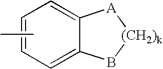

(in the formula, the symbols are as described above).

10. A therapeutic agent of allergic disease as described in the fifth through ninth aspects, where W is —O—; and R² is —OR⁶ (where R⁶ is hydrogen atom or alkyl).

11. A therapeutic agent of allergic disease as described in the fifth through tenth aspects, where the position for the substitution with WR¹ is position “j” in the benzene ring and the position for the substitution with R² is the position “i” in the benzene ring.

12. A therapeutic agent of allergic disease as described in the fifth and sixth aspects or the eighth through eleventh aspects, where Alk^{a} is alkylene and r=1.

13. A therapeutic agent of allergic disease as described in the fifth aspect, where the 2-oxoquinoline compound is selected from the group consisting of 7-methoxy-2-oxo-8-pentyloxy-1,2-dihydroquinoline-3-carboxylic acid (2-pyridin-4-ylethyl)amide, 7-methoxy-2-oxo-8-pentyloxy-1,2-dihydroquinoline-3-carboxylic acid (4-aminobenzyl)amide, 7-methoxy-2-oxo-8-pentyloxy-1,2-dihydroquinoline-3-carboxylic acid [2-(4-aminophenyl)ethyl]amide, 7-methoxy-2-oxo-8-pentyloxy-1,2-dihydroquinoline-3-carboxylic acid (4-aminophenyl)amide hydrochloride salt, 7-methoxy-2-oxo-8-pentyloxy-1,2-dihydroquinoline-3-carboxylic acid (3,4-methylenedioxybenzyl)amide, 8-ethoxy-7-methoxy-2-oxo-1,2-dihydroquinoline-3-carboxylic acid (2-pyridin-4-ylethyl)amide, 7-methoxy-2-oxo-8-pentyloxy-1,2-dihydroquinoline-3-carboxylic acid [2-(4-hydroxyphenyl)ethyl]amide, 7-methoxy-2-oxo-8-pentyloxy-1,2-dihydroquinoline-3-carboxylic acid [2-(4-fluorophenyl)ethyl]amide, 7-methoxy-2-oxo-8-pentyloxy-1,2-dihydroquinoline-3-carboxylic acid (4-pyridylmethyl)amide, 7-methoxy-2-oxo-8-pentyloxy-1,2-dihydroquinoline-3-carboxylic acid (2-piperidinoethyl)amide, 7-methoxy-2-oxo-8-pentyloxy-1,2-dihydroquinoline-3-carboxylic acid (2-morpholinoethyl)amide, 7-methoxy-2-oxo-8-pentyloxy-1,2-dihydroquinoline-3-carboxylic acid (3-pyridylmethyl)amide, 7-methoxy-2-oxo-8-pentyloxy-1,2-dihydroquinoline-3-carboxylic acid (2-pyridylmethyl)amide, 8-butoxy-7-methoxy-2-oxo-1,2-dihydroquinoline-3-carboxylic acid (2-phenylethyl)amide, 8-butoxy-7-methoxy-2-oxo-1,2-dihydroquinoline-3-carboxylic acid [2-(4-fluorophenyl)ethyl]amide, 8-butoxy-7-methoxy-2-oxo-1,2-dihydroquinoline-3-carboxylic acid (2-pyridin-4-ylethyl)amide, 8-butoxy-7-methoxy-2-oxo-1,2-dihydroquinoline-3-carboxylic acid (2-pyridin-4-ylethyl)amide hydrochloride salt, 8-ethoxy-7-methoxy-2-oxo-1,2-dihydroquinoline-3-carboxylic acid [2-(4-fluorophenyl)ethyl]amide, 7-methoxy-2-oxo-8-pentyloxy-1,2-dihydroquinoline-3-carboxylic acid [2-(2-fluorophenyl)ethyl]amide, 7-methoxy-2-oxo-8-pentyloxy-1,2-dihydroquinoline-3-carboxylic acid [2-(3-fluorophenyl)ethyl]amide, 7-methoxy-2-oxo-8-pentyloxy-1,2-dihydroquinoline-3-carboxylic acid [2-(4-hydroxy-3-methoxyphenyl)ethyl]amide, 7-methoxy-2-oxo-8-pentyloxy-1,2-dihydroquinoline-3-carboxylic acid [2-(4-chrolophenyl)ethyl]amide, 7-methoxy-2-oxo-8-pentyloxy-1,2-dihydroquinoline-3-carboxylic acid (2-phenyl)ethylamide, 7-methoxy-2-oxo-8-pentyloxy-1,2-dihydroquinoline-3-carboxylic acid (4-methylbenzyl)amide, 7-methoxy-2-oxo-8-pentyloxy-1,2-dihydroquinoline-3-carboxylic acid (4-fluorobenzyl)amide, 7-methoxy-2-oxo-8-propoxy-1,2-dihydroquinoline-3-carboxylic acid (2-pyridin-4-ylethyl)amide, 7-methoxy-2-oxo-8-propoxy-1,2-dihydroquinoline-3-carboxylic acid[2-(4-fluorophenyl)ethyl)]amide, 7-methoxy-2-oxo-8-propoxy-1,2-dihydroquinoline-3-carboxylic acid[2-(4-hydroxyphenyl)ethyl]amide, 7-methoxy-2-oxo-8-propoxy-1,2-dihydroquinoline-3-carboxylic acid (3,4-methylenedioxybenzyl)amide, 7-methoxy-2-oxo-8-propoxy-1,2-dihydroquinoline-3-carboxylic acid (2-phenylethyl)amide, 7,8-dimethoxy-2-oxo-1,2-dihydroquinoline-3-carboxylic acid [2-(4-fluorophenyl)ethyl)]amide, 7-methoxy-2-oxo-6-pentyloxy-1,2-dihydroquinoline-3-carboxylic acid [2-(4-fluorophenyl)ethyl]amide, 7-methoxy-2-oxo-6-pentyloxy-1,2-dihydroquinoline-3-carboxylic acid (3,4-methylenedioxybenzyl)amide, 7-methoxy-2-oxo-6-pentyloxy-1,2-dihydroquinoline-3-carboxylic acid (2-morpholinoethyl)amide, 8-ethoxy-7-methoxy-2-oxo-1,2-dihydroquinoline-3-carboxylic acid (3,4-methylenedioxybenzyl)amide, 1-methyl-7-methoxy-2-oxo-8-pentyloxy-1,2-dihydroquinoline-3-carboxylic acid [2-(4-fluorophenyl)ethyl]amide, 1-methyl-7-methoxy-2-oxo-8-pentyloxy-1,2-dihydroquinoline-3-carboxylic acid [2-pyridin-4-ylethyl]amide, 1-methyl-7-methoxy-2-oxo-8-pentyloxy-1,2-dihydroquinoline-3-carboxylic acid (2-morpholinoethyl)amide, 1-methyl-7-methoxy-2-oxo-8-pentyloxy-1,2-dihydroquinoline-3-carboxylic acid (4-pyridylmethyl)amide, 1-methyl-7-methoxy-2-oxo-8-pentyloxy-1,2-dihydroquinoline-3-carboxylic acid (4-fluorobenzyl)amide, 1-methyl-7-methoxy-2-oxo-8-pentyloxy-1,2-dihydroquinoline-3-carboxylic acid [2-(4-hydroxyphenyl)ethyl]amide, 1-methyl-7-methoxy-2-oxo-8-pentyloxy-1,2-dihydroquinoline-3-carboxylic acid (3,4-methylenedioxybenzyl)amide, 1-methyl-7-methoxy-2-oxo-6-pentyloxy-1,2-dihydroquinoline-3-carboxylic acid [2-(4-fluorophenyl)ethyl]amide, 1-methyl-7-methoxy-2-oxo-6-pentyloxy-1,2-dihydroquinoline-3-carboxylic acid (2-morpholinoethyl)amide, 1-methyl-7-methoxy-2-oxo-6-pentyloxy-1,2-dihydroquinoline-3-carboxylic acid (3,4-methylenedioxybenzyl)amide, 7,8-dipentyloxy-2-oxo-1,2-dihydroquinoline-3-carboxylic acid [2-(4-fluorophenyl)ethyl]amide, 8-hydroxy-7-methoxy-2-oxo-1,2-dihydroquinoline-3-carboxylic acid (3,4-methylenedioxybenzyl)amide, 7-methoxy-2-oxo-8-pentyloxy-1,2-dihydroquinoline-3-carboxylic acid (3,4-dihydroxybenzyl)amide, 7-methoxy-2-oxo-8-pentyloxy-1,2-dihydroquinoline-3-carboxylic acid (4-hydroxy-3-methoxybenzyl)amide, 1-O-{2-hydroxy-5-[(7-methoxy-2-oxo-8-pentylxoy-1,2-dihydro-3-quinolyl)carbonylaminomethyl]phenyl}glucoside uronic acid and 1-O-{2-hydroxy-4-[(7-methoxy-2-oxo-8-pentylxoy-1,2-dihydro-3-quinolyl)carbonylaminomethyl]phenyl}glucoside uronic acid, 5-[(7-methoxy-3-[(3,4-methylenedioxybenzyl)carbamoyl]-2-oxo-1,2-dihydro-8-quinolyloxy)pentanoic acid, 5-[(7-methoxy-3-[(3-hydroxy-4-methoxybenzyl)carbamoyl]-2-oxo-1,2-dihydro-8-quinolyloxy)pentanoic acid, 8-(5-hydroxypentyloxy)-7-methoxy-2-oxo-1,2-dihydroquinoline-3-carboxylic acid (3,4-methylenedioxybenzyl)amide, 8-(5-hydroxypentyloxy)-7-methoxy-2-oxo-1,2-dihydroquinoline-3-carboxylic acid (4-hydroxy-3-methoxybenzyl)amide, 8-(4-hydroxypentyloxy)-7-methoxy-2-oxo-1,2-dihydroquinoline-3-carboxylic acid (3,4-methylenedioxybenzyl)amide, 7-methoxy-2-oxo-8-(4-oxopentyloxy)-1,2-dihydroquinoline-3-carboxylic acid (3,4-methylenedioxybenzyl)amide, 8-(3-hydroxypentyloxy)-7-methoxy-2-oxo-1,2-dihydroquinoline-3-carboxylic acid (3,4-methylenedioxybenzyl)amide, 7-methoxy-2-oxo-8-(3-oxopentyloxy)-1,2-dihydroquinoline-3-carboxylic acid (3,4-methylenedioxybenzyl)amide, 8-(2-hydroxypentyloxy)-7-methoxy-2-oxo-1,2-dihydroquinoline-3-carboxylic acid (3,4-methylenedioxybenzyl)amide, 7,8-dihydroxy-2-oxo-1,2-dihydroquinoline-3-carboxylic acid [2-(4-fluorophenyl)ethyl]amide, 8-butoxy-3-hydroxymethyl-7-methoxy-2-oxo-1,2-dihydroquinoline, 8-ethoxy-3-hydroxymethyl-7-methoxy-2-oxo-1,2-dihydroquinoline, N-(4-fluorophenyl)carbamic acid (8-butoxy-7-methoxy-2-oxo-1,2-dihydroquinolin-3-yl) methyl ester, N-pyridin-4-ylcarbamic acid (8-ethoxy-7-methoxy-2-oxo-1,2-dihydroquinolin-3-yl)methyl ester, 3-dimethylaminomethyl-8-ethoxy-7-methoxy-2-oxo-1,2-dihydroquinoline, 8-butoxy-3-aminomethyl-7-methoxy-2-oxo-1,2-dihydroquinoline, 8-ethoxy-7-methoxy-3-morpholinomethyl-2-oxo-1,2-dihydroquinoline, N-[(8-butoxy-7-methoxy-2-oxo-1,2-dihydroquinolin-3-yl)methyl]-N′-(4-fluorophenyl) urea and N-[(8-butoxy-7-methoxy-2-oxo-1,2-dihydroquinolin-3-yl)methyl]-(4-hydroxyphenyl)acetamide.

14. A therapeutic agent of allergic disease as described in the third aspect, where the cannabinoid receptor-regulating substance is N-(benzo[1,3]dioxol-5-ylmethyl)-7-methoxy-2-oxo-8-pentyloxy-1,2-dihydroquinoline-3-carboxamide or a pharmaceutically acceptable salt thereof.

15. A therapeutic agent of allergic disease as described in the first or second aspect, where the cannabinoid receptor-regulating substance is selected from a group consisting of SR144528, HU-308, L-759 633, L-759 656, L-768 242, PRS-211 096, PRS-211 335, PRS-211 359, AM603, AM1703, AM1710 and AM1221.

16. A therapeutic agent of allergic disease as described in the fifteenth aspect, where the cannabinoid receptor-regulating substance is SR144528.

17. A therapeutic agent of allergic disease as described in the first through sixteenth aspects, where the cannabinoid receptor-regulating substance is an inverse agonist.

18. A therapeutic agent of allergic disease as described in the first through seventeenth aspects, where the cannabinoid receptor-regulating substance is a substance increasing the amount of cAMP generated via an adenylate cyclase-activating agent.

19. A therapeutic agent of allergic disease as described in the first through seventeenth aspects, where the cannabinoid receptor-regulating substance is an inverse agonist selective for peripheral cell type cannabinoid receptor.

20. A therapeutic agent of allergic disease as described in the first through nineteenth aspects, where the allergic disease is allergic dermatitis.

21. A therapeutic agent of allergic disease as described in the first through twentieth aspects, where the allergic disease is atopic dermatitis.

22. A therapeutic agent of allergic disease as described in the first through nineteenth aspects, where the allergic disease is allergic asthma.

23. A therapeutic agent of allergic disease as described in the first through nineteenth aspects or the twenty-second aspect, where the allergic disease is early phase asthma response or/and late phase asthma response or/and airway hypersensitivity.

24. A therapeutic agent of allergic disease as described in the first through nineteenth aspects, where the allergic disease is allergic rhinitis or allergic conjunctivitis.

25. A therapeutic agent of allergic disease as described in the first through twenty-fourth aspects, where the cannabinoid receptor-regulating substance concurrently has an action inhibiting leukotriene.

26. A therapeutic agent of allergic disease as described in the first through twenty-fifth aspects, where the therapeutic agent shows a therapeutic effect in animals with DNFB-induced allergic dermatitis or in animals with IgE-dependent allergic dermatitis.

27. A pharmaceutical agent of a cannabinoid receptor-regulating substance for the therapeutic treatment of allergic disease due to cannabinoid agonist.

28. A method for identifying an anti-allergic agent, including the following steps:

1. a step of selecting compounds selectively binding to peripheral cell type cannabinoid receptor,

2. a step of selecting a compound as an inverse agonist from the compounds selected at the step 1 or compounds known to selectively bind to peripheral cell type cannabinoid receptor, and

3. a step of measuring the anti-allergic effect of the compound selected at the step 2.

29. A therapeutic agent of allergic disease, the therapeutic agent containing as the active ingredient N-(benzo[1,3]dioxol-5-ylmethyl)-7-methoxy-2-oxo-8-pentyloxy-1,2-dihydroquinoline-3-carboxamide or a pharmaceutically acceptable salt thereof.

30. A therapeutic agent of atopic dermatitis, the therapeutic agent containing as the active ingredient N-(benzo[1,3]dioxol-5-ylmethyl)-7-methoxy-2-oxo-8-pentyloxy-1,2-dihydroquinoline-3-carboxamide or a pharmaceutically acceptable salt thereof.

31. A therapeutic agent of asthma, the therapeutic agent containing as the active ingredient N-(benzo[1,3]dioxol-5-ylmethyl)-7-methoxy-2-oxo-8-pentyloxy-1,2-dihydroquinoline-3-carboxamide or a pharmaceutically acceptable salt thereof.

### BRIEF DESCRIPTION OF THE DRAWINGS

FIG. 1 shows the graph depicting the influence of the test compound on ear swelling after a fourth antigen application in murine DNFB-induced allergic dermatitis model. The axis of ordinate expresses “the increment (×10⁻² mm) of the thickness of ear” in the mouse before and after the dosing of the test compound. The results of shame treatment, vehicle (oral dosing of solvent at 10 mg/kg alone), prednisolone (oral dosing of 1, 2 and 5 mg/kg as positive control), and Compound A (oral dosing at 0.1, 1, 10 mg/kg) are shown from the left.

FIG. 2 shows the graph depicting the influence of the test compound on ear swelling after a fifth antigen application on murine DNFB-induced allergic dermatitis. The axis of ordinate expresses “the increment (×10⁻² mm) of the thickness of ear” in the mouse before and after the dosing of the test compound. The results of shame treatment, vehicle (oral dosing of solvent at 10 mg/kg alone), prednisolone (oral dosing of 1, 2 and 5 mg/kg as positive control), and Compound A (oral dosing at 0.1, 1, 10 mg/kg) are shown from the left.

FIG. 3 shows the graph depicting the influence of the test compound on the spleen wet weight in the DNFB-induced allergic dermatitis model. The axis of ordinate expresses the wet spleen weight (mg). The results of shame treatment, vehicle (oral dosing of solvent at 10 mg/kg alone), prednisolone (oral dosing of 1, 2 and 5 mg/kg as positive control), and Compound A (oral dosing at 0.1, 1, 10 mg/kg) are shown from the left.

FIG. 4 shows the graph depicting the influence of the test compound on ear swelling in the early phase (one hour after antigen application) of murine IgE-dependent allergic dermatitis reaction. The axis of ordinate expresses “the increment (×10⁻² mm) of the thickness of ear” in the mouse before and after the dosing of the test compound. The results of vehicle (oral dosing of vehicle at 10 mg/kg alone), ketotifen fumarate (oral dosing of 10 mg/kg as positive control), pranlukast hydrate (oral dosing at 30 mg/kg as positive control), and Compound A (oral dosing at 10 mg/kg) are shown from the left.

FIG. 5 shows the graph depicting the influence of the test compound on ear swelling in the late phase (24 hours after antigen application) of murine IgE-dependent allergic dermatitis reaction. The axis of ordinate expresses “the increment (×10⁻² mm) of the thickness of ear” in the mouse before and after the dosing of the test compound. The results of solvent (oral dosing of vehicle at 10 mg/kg alone), ketotifen fumarate (oral dosing of 10 mg/kg as positive control), pranlukast hydrate (oral dosing at 30 mg/kg as positive control), and Compound A (oral dosing at 10 mg/kg) are shown from the left.

FIG. 6 shows the graph depicting the influence of the test compound on ear swelling in the very late phase (8 days after antigen application) of murine IgE-dependent allergic dermatitis reaction. The axis of ordinate expresses “the increment (×10⁻² mm) of the thickness of ear” in the mouse before and after the dosing of the test compound. The results of solvent (oral dosing of vehicle at 10 mg/kg alone), ketotifen fumarate (oral dosing of 10 mg/kg as positive control), pranlukast hydrate (oral dosing at 30 mg/kg as positive control), and Compound A (oral dosing at 10 mg/kg) are shown from the left.

FIG. 7 shows the graph depicting the influence of the period of the dosing of the test compound in murine IgE-dependent allergic dermatitis reaction. The axis of ordinate expresses “the increment (×10⁻² mm) of the thickness of ear” in the mouse before and after the dosing of the test compound. The symbol 0-8 expresses the period of dosing from the antigen application day to 8 days later. The results of vehicle (oral dosing of vehicle at 10 mg/kg alone for 9 days), and Compound A (oral dosing at 10 mg/kg for 9, 8, 7, 5, 3 days) are shown from the left.

FIG. 8 shows the graph depicting the influence of the test compound on the respiratory resistance in the early phase asthma (one minute after antigen challenge) in antigen-induced asthma in guinea pigs. The axis of ordinate expresses the increment (%) of airway resistance (sRaw). The results of sham treatment, vehicle (oral dosing of solvent at 10 mg/kg alone), Compound A (oral dosing at 10, 30, 100 mg/kg), pranlukast (oral dosing at 30 mg/kg as positive control) and prednisolone (oral dosing at 30 mg/kg as positive control).

FIG. 9 shows the graph depicting the influence of the test compound on the respiratory resistance in the late phase asthma (4 to 8 hours after the antigen challenge) in antigen-induced asthma in guinea pig. The axis of ordinate expresses AUC_{4-8 hr} (%·hr). AUC_{4-8 hr} means the increment (area under the curve) of the airway resistance (sRaw) over 4 to 8 hours after the antigen challenge. The results of sham treatment, vehicle (oral dosing of solvent at 10 mg/kg alone), Compound A (oral dosing at 10, 30, 100 mg/kg), pranlukast (oral dosing at 30 mg/kg as positive control) and prednisolone (oral dosing at 30 mg/kg as positive control).

FIG. 10 shows the graph depicting the influence of the test compound on the airway hypersensitivity in guinea pigs. The axis of ordinate expresses PC₁₀₀ ACh (mg/ml). PC₁₀₀ ACh means the acetylcholine concentration required for airway resistance (sRaw) after acetylcholine inhalation to get 100% increment of the sRaw after physiological saline inhalation. The results of sham treatment, vehicle (oral, dosing of solvent at 10 mg/kg alone), Compound A (oral dosing at 10, 30, 100 mg/kg), pranlukast (oral dosing at 30 mg/kg as positive control) and prednisolone (oral dosing at 30 mg/kg as positive control) are shown from the left.

FIG. 11 shows the graph depicting the influence of the test compound on leukotriene generation from human basophils. The axis of ordinate expresses the amount (pg/mL) of leukotrienes (C4/D4/E4), while the axis of abscissa expresses the amount (μg/mL) of anti-IgE antibody.

FIG. 12 shows the graph depicting the influence of the test compound on leukotriene generation from rat mast cells. The axis of ordinate expresses the amount (pg/mL) of leukotrienes (C4/D4/E4), while the axis of abscissa expresses the amount (ng/mL) of DNP-BSA.

FIG. 13 shows the graph depicting the influence of the test compound on ear swelling in the very late phase (8 days after antigen application) in murine IgE-dependent allergic dermatitis reaction. The axis of ordinate expresses “the enlargement (×10⁻² mm) of the thickness of ear” in the mouse before and after the dosing of the test compound. The results of the non-sensitized group, the sensitized group, HU-308 (oral dosing of 10 and 50 mg/kg), Compound A (oral dosing at 0.1, 1, 10 mg/kg), and prednisolone (oral dosing of 5 mg/kg as positive control) are shown from the left of the above figure. The following figure individually shows the results of the non-sensitized group, the sensitized group, SR144528 (oral dosing of 0.1, 1 and 10 mg/kg), Compound A (oral dosing at 10 mg/kg), and prednisolone (oral dosing of 5 mg/kg as positive control) from the left.

**: p<0.01

***: P<0.001 (vs the sensitized group; Dunnett test)

###: p<0.001 (vs the sensitized group; Student's t test)

$$$: p<0.001 (vs the non-sensitized group; Student's t test)

FIG. 14 shows the graph depicting the influence of the test compound on the spleen wet weight and the thymus wet weight in the very late phase (8 days after antigen application) in the murine IgE-dependent allergic dermatitis model. The axis of ordinate in the above figure expresses the spleen wet weight (mg). The axis of ordinate in the following figure expresses the thymus wet weight (mg). The figures individually show the results of the non-sensitized group, the sensitized group, HU-308 (oral dosing of 10 and 50 mg/kg), Compound A (oral dosing at 0.1, 1, 10 mg/kg), and prednisolone (oral dosing of 5 mg/kg) from the left.

**: p<0.05

***: P<0.001 (vs the sensitized group; Dunnett test)

##: p<0.01

###: p<0.001 (vs the sensitized group; Student's t test)

$$: p<0.01 (vs the non-sensitized group; Student's t test)

FIG. 15 shows the graph depicting the influence of the test compound on the spleen wet weight and the thymus wet weight in the very late phase (8 days after antigen application) in the murine IgE-dependent allergic dermatitis model. The axis of ordinate in the above figure expresses the spleen wet weight (mg). The axis of ordinate in the following figure expresses the thymus wet weight (mg). The figures individually show the results of the non-sensitized group, the sensitized group, SR144528 (oral dosing of 0.1, 1 and 10 mg/kg), Compound A (oral dosing at 10 mg/kg), and prednisolone (oral dosing of 5 mg/kg) from the left.

###: P<0.001 (vs the sensitized group; Student's t test)

FIG. 16 depicts the change of ear swelling induced by the test compounds after application to mouse ear. The axis of ordinate expresses the increase (×10⁻² mm) of the thickness of ear in the mouse before and after the dosing of the test compound. The axis of abscissa expresses the time after the application of the compound from the left. Mean±standard (n=6)

FIG. 17 shows the graph depicting the effect of the Compound A on ear edema induced by 2-arachidonoylglycerol ether (2-AG-E) applied in the mice ear. The axis of ordinate expresses AUC (on day 0 to day 8). The graphs from the left show the results of sham treatment, vehicle (oral dosing of solvent at 10 mg/kg alone), and Compound A (oral dosing of 0.01, 0.1, 1 and 10 mg/kg). Mean±standard (n=8)

**: p<0.01

***: P<0.001 (vs the solvent group; Dunnett test)

$$$: p<0.001 (vs the false treatment group; Student's t test)

FIG. 18 shows the effect of the test compound on spontaneous scratching reaction in NC mouse. The axis of ordinate expresses the number of scratching movements (movements/hour) in the mouse before or after the dosing of the test compound. The graphs from the left show the results of vehicle (oral dosing of solvent alone), Compound A (oral dosing of 1 and 10 mg/kg), tacrolimus hydrate (oral dosing of 1 mg/kg) and betamethasone valerate (oral dosing of 1 mg/kg).

### DETAILED DESCRIPTION OF THE INVENTION

The terms to be used in the present specification are now described below.

The terms “cannabinoid receptor-regulating substance” and “cannabinoid receptor regulator” mean a substance regulating the biological activity of cannabinoid receptor or a substance regulating the expression of cannabinoid receptor. The former includes agonists, antagonists, inverse agonists, and other substances enhancing or reducing the sensitivity of cannabinoid receptor. The latter includes a substance enhancing or suppressing the expression of the gene of cannabinoid receptor. The inverse agonist has an action inverse to the original action of the agonist of the receptor. A finding has been obtained that in view of cAMP level for cannabinoid receptor, for example, Compound A increases the cAMP level while cannabinoid suppresses the increase of cAMP. Specifically, the inverse agonist includes Compound A, SR144528 and AM630, preferably Compound A and SR144528.

The cannabinoid receptor-regulating substance specifically includes for example compounds represented by the general formula [I] in JP-A-2000-256323 (WO 00/40562) and more specifically includes 2-oxoquinoline compounds such as N-(benzo[1,3]dioxol-5-ylmethyl)-7-methoxy-2-oxo-8-pentyloxy-1,2-dihydroquinoline-3-carboxamide (Compound A) and additionally Δ9-THC, Nabilone (LY-109514), CP-55940, PRS-211096, PRS-211335, PRS-211359, SR144528, SR141716, Rimonabant (SR141716A), SR14778, AMG-3, SLV-319, AM-251, AM-281, AM374, AM404, AM630, AM-694, AM2233, AM2230, compounds described in WO 01/28557 such as AM1221, compounds described in WO 01/28497 such as AM1703, compounds described in WO 01/28329 such as AM1710, compounds described in WO 01/32169 such as HU-308, compounds described in WO 99/51560 such as HU-310, JWH-051, JWH-161, 0-1236, 0-1057, 0-2093, L-759633, L-759656, L-768242, compounds described in WO 96/02248 such as LY320135, BAY-38-7221, compounds described in WO 02/24630, compounds described in WO 02/10135, compounds described in WO 01/96330, compounds described in WO 01/85092, compounds described in WO 01/74763, compounds described in WO 01/70700, compounds described in WO 01/64634, compounds described in WO 01/64633, compounds described in WO 01/64632, compounds described in WO 01/58869, compounds described in WO 01/58445, compounds described in WO 01/04083, compounds described in WO 01/32629, compounds described in WO 01/29007, compounds described in WO 01/28588, compounds described in JP-T-2001-515470 (U.S. Pat. No. 6 262 112), compounds described in JP-T-2002-539246 (WO 00/56303), compounds described in WO 00/46209, compounds described in WO 00/32200, compounds described in WO 00/16756, compounds described in WO 00/15609, compounds described in JP-T-2002-523396 (WO 00/10968), compounds described in JP-T-2002-523395 (WO 00/10967), compounds described in WO 99/60987, compounds described in WO 99/57107, compounds described in WO 99/57106, compounds described in WO 99/52524, compounds described in WO 99/26612, compounds described in WO 99/24471, compounds described in WO 99/2499, compounds described in JP-T-2001-516361 (WO 98/41519), compounds described in WO 98/37061, compounds described in WO 98/32441, JP-T-2001-508799 (WO 98/31227), compounds described in WO 97/29079, compounds described in JP-T-2000-502080 (WO 97/21682), compound described in WO 97/19063, compounds described in JP-T-11-507937 (WO 97/860), compounds described in WO 96/20268, compounds described in JP-T-10-508870 (WO 96/25397), compounds described in JP-T-11-501615 (WO 96/18600), compounds described in JP-T-11-500411 (WO 96/18391), compounds described in WO 94/12466, compounds described in U.S. Pat. No. 6,284,788, compounds described in U.S. Pat. No. 5,939,429, compounds described in U.S. Pat. No. 5,804,592, compounds described in U.S. Pat. No. 5,605,906, compounds described in U.S. Pat. No. 5,624,941, compounds described in U.S. Pat. No. 5,462,960, compounds described in U.S. Pat. No. 5,081,122, compounds described in U.S. Pat. No. 5,013,837, compounds described in DE 10015866, compounds described in DE 19837627, compounds described in DE 19837638, compounds described in WO 01/58450, compounds described in WO 01/32663, compounds described in WO 01/28498, compounds described in WO 01/24798, compounds described in FR 2805818, compounds described in 2805817, compounds described in FR 2805810, compounds described in 279912, compounds described in FR 2789079, compounds described in FR 2789078, compounds described in WO 01/89589, compounds described in WO 01/95889, compounds described in WO 01/98289, compounds described in WO 02/19383, compounds described in WO 02/26702, compounds described in WO 02/28346, compounds described in WO 01/87297, compounds described in WO 02/36590, compounds described in WO 02/42269, compounds described in WO 02/42248, compounds described in WO 02/47691, compounds described in WO 02/58636, compounds described in WO 02/60447, compounds described in W 02/65997, compounds described in WO 02/53543, compounds described in WO 02/72562, compounds described in WO 02/62750, compounds described in WO 02/80903, and compounds described in WO 02/85866.

Preferably, the cannabinoid receptor-regulating substance is a regulating substance selectively acting on peripheral cell type cannabinoid receptor, such as the compounds described in JP-A-2000-256323 (WO 00/40562), the compounds described in WO 02/10135, SR 144528, AM 630, the compounds described in WO 01/28557 such as AM1221, compounds described in WO 01/28497 such as AM1703, compounds described in WO 01/28329 such as AM1710, compounds described in WO 01/32169 such as HU-308, JWH-051, L-759633, L-759656, L-768242, compounds described in WO 01/74763, compounds described in WO 01/32629, compounds described in WO 01/29007, compounds described in WO 01/19807, compounds described in WO 01/4083, compounds described in JP-T-2002-539246 (WO 00/56303), compounds described in JP-T-2002-523396 (WO 00/10968), compounds described in JP-T-2002-523395 (WO 00/10967), compounds described in WO 99/57107, compounds described in WO 99/2499, compounds described in JP-T-2001-516361 (WO 98/41519), compounds described in JP-T-2001-515470 (US 6262112), compounds described in JP-T-2001-508799 (WO 98/31227), compounds described in WO 97/29079, compounds described in JP-T-2000-502080 (WO97/21682), compounds described in JP-T-11-507937 (WO 97/860), compounds described in JP-T-10-508870 (WO 96/25397), compounds described in JP-T-11-501615 (WO 96/18600), compounds described in JP-T-11-500411 (WO 96/18391), compounds described in U.S. Pat. No. 5,605,906, compounds described in WO 01/58869, compounds described in WO 01/96330, compounds described in WO 02/10135, compounds described in WO 02/42269, compounds described in WO 02/58636, compounds described in WO 02/60447, compounds described in W 02/53543, compounds described in WO 02/72562, compounds described in WO 02/62750, and compounds described in WO 02/85866, and still more preferably includes the compounds described in JP-A-2000-256323 (WO 00/40562), SR144528, AM 630, the compounds described in WO 01/28557 such as AM1221, the compounds described in WO 01/28497 such as AM1703, the compounds described in WO 01/28329 such as AM1710, the compounds described in WO 01/32169 such as HU-308, JWH-051, L-759633, L-759656, L-768242, the compounds described in WO 01/32629, the compounds described in WO 01/29007, and the compounds described in WO 98/41519. Particularly preferably, the 2-oxoquinoline compound includes N-(benzo[1,3]dioxol-5-ylmethyl)-7-methoxy-2-oxo-8-pentyloxy-1,2-dihydroquinoline-3-carboxamide (Compound A). More preferably, the 2-oxoquinoline compound includes Compound A, SR144528, AM630. Still more preferably, the compound includes Compound A and SR144528. Most preferably, the compound is Compound A.

The “allergic disease” includes but is not limited to anaphylaxis, digestive tract allergy, allergic gastritis, allergic dermatitis, dermatitis such as rash against Japanese lacquer (urushi) and rash against cosmetics, urticaria, atopic dermatitis, asthma, allergic asthma, atopic asthma, allergic bronchial pulmoaspergillosis, pollenosis, allergic rhinitis, allergic conjunctivitis, allergic sarcoma angitis, chemical allergy, serum disease, post-organ grafting rejection, tuberculosis lesion, and post-organ grafting rejection. The term allergic disease is applicable to any disease in relation with allergy. More preferably, the allergic disease includes allergic dermatitis, atopic dermatitis, asthma, allergic asthma, atopic asthma, allergic rhinitis, and allergic conjunctivitis. Particularly preferably, the allergic disease includes allergic disease of skin or respiratory organ. More specific indication includes allergic dermatitis, atopic dermatitis, allergic asthma and atopic asthma.

The term “allergic dermatitis” means dermatitis in relation with allergic reaction and includes for example atopic dermatitis. The allergic dermatitis is discriminated from non-allergic dermatitis such as dermatitis due to injuries or wounds. The “therapeutic agent of atopic dermatitis” preferably can have enhanced therapeutic effect via the action thereof on the allergic reaction occurring in atopic dermatitis. Further, the therapeutic agent of atopic dermatitis preferably has an effect on the late phase response of the allergic reaction, the very late phase response (delayed response) thereof, or the late phase response thereof and very late phase response thereof. More preferably, the therapeutic agent of atopic dermatitis has an effect on the late phase response, the very late phase response, or the late phase response and the very late phase response, in addition to the early phase response.

The term “allergic asthma” means an allergic aspect of asthma among asthma symptoms and includes for example mixed type asthma and atopic asthma, which is discriminated from non-allergic asthma such as aspirin asthma. The “therapeutic agent of asthma” preferably exerts a therapeutic effect via the action on the allergic reaction of asthma. Further, the therapeutic agent of asthma preferably exerts an effect on chronic bronchitis or airway hypersensitivity. More preferably, the therapeutic agent of asthma has an effect on chronic bronchitis and airway hypersensitivity. Additionally, the therapeutic agent of asthma exerts an effect on the late phase response of the allergic reaction, the very late phase response thereof, or the late phase response and the very late phase response. Still more preferably, the therapeutic agent of asthma exerts an effect on the late phase response reaction, the very late phase response, or the late phase response reaction and the very late phase response, in addition to the early phase response.

The term “itching-soothing action” means to reduce scratching action to reduce psychological stress due to itching, by reducing itching or eliminating itching. Preferably, the causes of itching are eliminated such as anti-histamine action or anti-substance P action, not by central action. Further, the therapeutic agent preferably has an itching-soothing action for allergic disease, particularly atopic dermatitis.

The “cannabinoid receptor-regulating substance” of the invention may possibly be a safe pharmaceutical agent without “immunosuppressive action as side effect”, as is observed in the case of steroidal agents and immunosuppressive agents. The “immunosuppressive action as side effect” includes hyperkalemia, leucopenia and thrombocytopenia due to the functional disorders of kidney and spleen, for which the decrease of spleen weight works as an indicator. No such side effect cannot be observed for the “cannabinoid receptor-regulating substance” of the invention.

Compared with ointments and injections and the like, a pharmaceutical agent possibly dosed orally can be handled easily, because the pharmaceutical agent has no distinct side effect.

Herein, the “therapeutic treatment” of allergic disease means suppression of allergic reaction or amelioration of allergic symptoms and includes the prevention of potential allergic reactions or allergic disease and the prevention of the exacerbation thereof.

### DESCRIPTION OF THE PREFERRED EMBODIMENTS

The invention is now described below in the following Examples. However, the invention is not limited to them.

### PREPARATION EXAMPLE 1

Production of Capsule

| | | |
|---|---|---|
| 1. | Compound A | 30 g |
| 2. | Particle cellulose | 10 g |
| 3. | Lactose | 19 g |
| 4. | Magnesium stearate | 1 g |
| Total | | 60 g |

The ingredients 1 through 4 are mixed together and charged in a gelatin capsule.

### PREPARATION EXAMPLE 2

Production of Tablet

| | | |
|---|---|---|
| 1. | Compound A | 30 g |
| 2. | lactose | 50 g |
| 3. | Corn starch | 15 g |
| 4. | Carboxymethyl cellulose calcium | 44 g |
| 5. | Magnesium stearate | 1 g |
| Total | | 140 g |

The ingredients 1 through 3 of their total weights and 30 g of the ingredient 4 are kneaded with water, dried in vacuum and prepared into granule. 14 g of the ingredient 4 and the ingredient 5 are mixed into the resulting granule, which is then tableted with a tableting machine. 1000 tablets are recovered, each tablet containing 30 mg of Compound A.

In case that the compound of the invention is to be used as a pharmaceutical composition, the compound itself can be dosed directly to patients. Additionally, the compound may be formulated into a preparation by known pharmaceutical methods, for dosing. If necessary, for example, the compound can be dosed orally or parenterally as microcapsules, soft and hard capsules, pills, liquids, powders, granules, fine granules, film coating preparations, pellets, troches, sublingual preparations, chewing preparations, buccal preparations, pastes, syrups, suspensions, elixirs, emulsions, eye drops, ear drops, coating preparations, ointments, hard ointments, cataplasm, TTS preparations, lotions, aspiration preparations, and aerosol, or parenterally as sterile solutions with water or pharmacologically acceptable solutions except for water or as injections of suspended and/or emulsified solutions, in addition to the Preparation Example 1 (capsule) and the Preparation Example 2 (tablet). Other forms for parenteral dosing include external liquid preparations and suppositories, pessaries and emulsion foaming agents for enteric dosing, which contain one or more active substances and are prepared by routine methods. Furthermore, the compound is appropriately combined and mixed for example with pharmacologically acceptable carriers or media, specifically including sterile water and physiological saline, vegetable oil, solvent, base, emulsifier, suspending agent, surfactant, stabilizer, flavor, aromatic agent, excipient, vehicle, preservative, binder, diluent, isotonic agent, soothing agent, filler, disintegrator, buffer, coating agent, lubricant, coloring agent, sweetener, viscous agent, corrigent, solubilizer, other additives and the like, in the form of unit dose according to generally accepted pharmaceutical practice, to form preparations.

Additives to be mixed into tablets and capsules include for example binders such as gelatin, corn starch, tragacanth gum, and gum arabic; excipients such as crystal cellulose; expansion agents such as corn starch, gelatin and alginic acid; lubricants such as magnesium stearate; sweeteners such as sucrose, lactose or saccharin; and flavors such as peppermint, Gaultheria adenothris oil or cherry. When the form of unit dose for preparation is capsule, the capsule can contain liquid carriers such as fats and oils in addition to the aforementioned materials. The sterile composition for injections can be prepared according to general pharmaceutical practice using the vihicle such as distilled water for injections.

The aqueous solution for injections includes for example isotonic solutions containing physiological saline, glucose and other auxiliary agents, for example D-sorbitol, D-mannose, D-mannitol, and sodium chloride, which may be used in combination with for example solubilizers for example alcohol, specifically including ethanol, polyalcohol for example propylene glycol and polyethylene glycol, and nonionic surfactants such as polysorbate 80 (TM) and HCO-50.

The oily liquid includes for example sesame seed oil and soybean oil. The solubilizer includes for example benzyl benzoate and benzyl alcohol. Further, the compound may satisfactorily be blended with buffers for example phosphate buffer and sodium acetate buffer, soothing agents for example procaine hydrochloride, stabilizers for example benzyl alcohol and phenol, and anti-oxidants. The prepared injections are generally filled in appropriate capsules.

The dose may vary, depending on the type and severity of a disease, the compound to be dosed and the dosing route, the age, sex, body weight, etc. of a patient, and the like. In case of oral dosing, generally, Compound A is dosed at 0.1 to 1,000 mg, preferably 1 to 300 mg per day per adult in one dose or in dividend portions.

Further, the compound of the invention is applicable as pharmaceutical agents for animals.

Pharmacological Tests

1. Therapeutic Effect in Allergic Dermatitis Model Animals

Atopic dermatitis is suggested as a complex disorder of Type I and Type IV allergic reactions. A model where Type I and Type IV allergic reactions occur singly or in combination is useful.

1. Effect on Murine DNFB-Induced Allergic Dermatitis

The model is produced by repeating antigen sensitization and induction in mouse to induce contact dermatitis involving the increase of IgE antibody titer, namely an inflammation similar to atopic dermatitis (J. Allergy Clin. Immunol., 100 (6Pt2), 39-44, December 1997). In the model, it is suggested that inflammation occurs via the delayed type allergic reaction via T cell and the late phase allergic response via mast cells. Simultaneously at the test, further, the spleen weight was measured so as to examine the systemic immunosuppressive action of a test compound.

Test Method

*Preparation of Test Compound

Solvent preparation: Methyl cellulose (referred to as MC) was dissolved in distilled water, to prepare aqueous 0.5% (w/v) MC solution.

Preparation of test compound: According to the Examples 3 to 5 of JP-A-2000-256323, a given amount of the Compound A was suspended in the solvent to prepare a 1 mg/ml suspension. By dilution, further, 0.1 mg/ml and 0.01 mg/ml suspensions were prepared. As a positive control, additionally, prednisolone (Sigma) was similarly prepared at 0.5 mg/ml, 0.2 mg/ml, and 0.1 mg/ml solutions. Prednisolone is one of adrenocorticosteroids approved of the efficacy for the therapeutic treatment of atopic dermatitis.

* Preparation and Application of Antigen

Antigen preparation: DNFB (2,4-dinitrofluorobenzene) was prepared with a mix solution of acetone and olive oil (3:1, v/v) to 0.15% (w/v) on a needed basis.

Antigen application: 25 μl each of the antigen solutions was applied on the surface and back surface of both the ears of a 9-week-old female BALB/c mouse (manufactured by SLC) once per week in total of 5 times.

* Dosing of Test Compound

In a period from the next day of the third antigen application to the next day of the fifth antigen application, the test compound was dosed at 10 mL/kg once a day in total of 15 times. Herein, the test compound was dosed one hour before the antigen application on the day of antigen application while the test compound was dosed 23 hours after the antigen application on the next day of the antigen application.

* Measurement of Ear Swelling

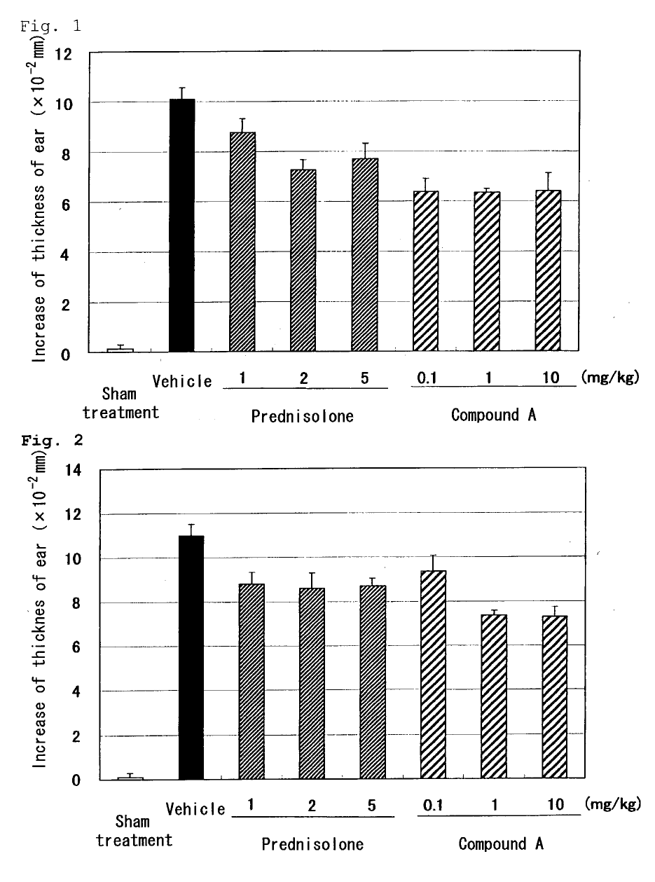

Before and 24 hours after the antigen application, the thickness of ear was measured with a dial thickness gauge (Yamazen Kikou). The difference was used as a parameter of swelling. FIGS. 1 and 2 show the results of the measurement at the fourth antigen application and the fifth antigen application, concurrently with the results of the positive control.

* Measurement of Spleen Weight

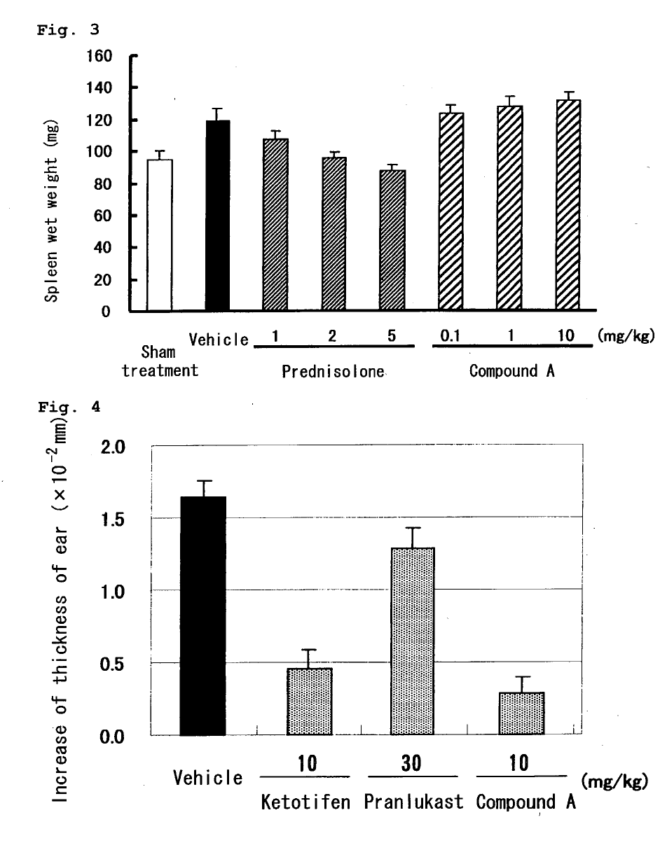

24 hours after the fifth antigen application, spleen was resected from the mouse exsanguinated under anesthesia with ether. The wet weight was measured. The results are shown in FIG. 3.

* Results

Nagai et al. report that ear swelling in a complex of the late phase response (Type I allergic reaction) and the very late phase response (Type IV allergic reaction) was developed after the fifth antigen application in the model.

The Compound A significantly suppressed ear swelling in the allergic dermatitis model. Additionally, the Compound A showed its effect at the start of dosing after the third antigen application. Then, the decrease of spleen weight as observed in the case of prednisolone was not observed.

2. Effect on Murine IgE-Dependent Allergic Dermatitis

The model was produced by passive sensitization of a mouse with IgE and repetition of antigen challenge to trigger triphase (early phase, late phase and very late phase) dermatitis (Pharmacology, 60(2), 97-104, February 2000). It has been verified that mast cells and T cells are involved in these reactions and eosinophils invades in local inflammatory sites. Thus, it is suggested that these reactions reflect a part of atopic dermatitis symptoms.

Test Method

* Preparation of Test Compound

Solvent preparation: MC was dissolved in distilled water, to prepare aqueous 0.5% MC solution.

Preparation of test compound: A given amount of the Compound A was suspended in the solvent to prepare 1 mg/ml suspension.

As positive controls, additionally, ketotifen fumarate (Sigma) of 1 mg/mL and pranlukast hydrate (extracted from Onon under trade name (Ono Pharmaceutical Co., Ltd.)) of 3 mg/mL were prepared. Pranlukast hydrate is used as a leukotriene inhibitor for therapeutic agents of asthma therapy and therapeutic agents of allergic rhinitis. Ketotifen fumarate is used as a suppressor of chemical mediator release for asthma, allergic rhinitis, eczema, dermatitis, urticaria, dermal pruritis, and allergic conjunctivitis.

* Passive Sensitization

Anti-DNP IgE (antibody against DNP; Yamasa Corporation) was prepared to 15 μg/mL with physiological saline. 0.2 mL of the resulting solution was dosed via caudal vein to female BALB/c mouse of age 9 weeks (manufactured by SLC).

* Antigen Preparation and Application

Antigen preparation: DNFB (2,4-dinitrofluorobenzene) was prepared with a mixed solution of acetone and olive oil (3:1, v/v) to 0.15% (w/v) on a needed basis.

* Antigen application: 24 hours after the dosing of the anti-DNP IgE, 25 μl each of the antigen solution was applied on the surface and back surface of both the ears.

* Dosing of Test Compound

From the antigen application day up to the day 8 after the antigen application, the test compound was orally dosed at 10 mL/kg once a day in total of 9 times. To other mice, herein, the test compound was orally dosed at 10 mL/kg once a day in total of 8 times from one day after the antigen application up to the day 8 of the antigen application. To other mice, similarly, the test compound was orally dosed at 10 mL/kg once a day in total of 7, 5 or 3 times from two days, four days or six days after the antigen application up to the day 8 after the antigen application. In the period from the antigen application day to the start of the dosing of the test compound, only the solvent in place of the test compound was orally dosed at 10 ml/kg once a day. Further, one hour before the antigen application on the day of antigen application and one hour before the measurement of the thickness of ear on the day 8 of the antigen dosing, the test compound was dosed.

* Measurement of Ear Swelling

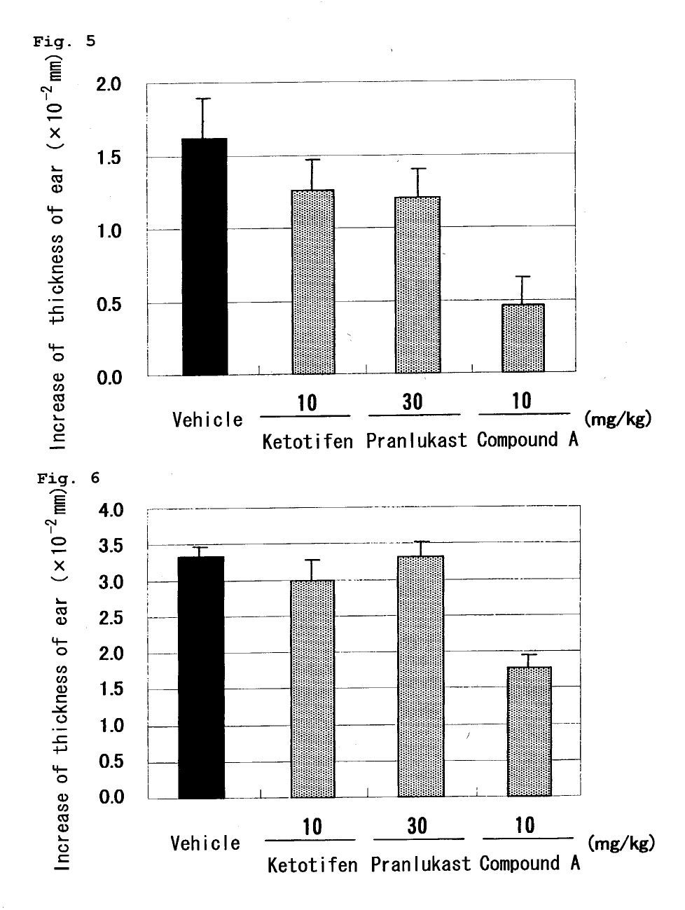

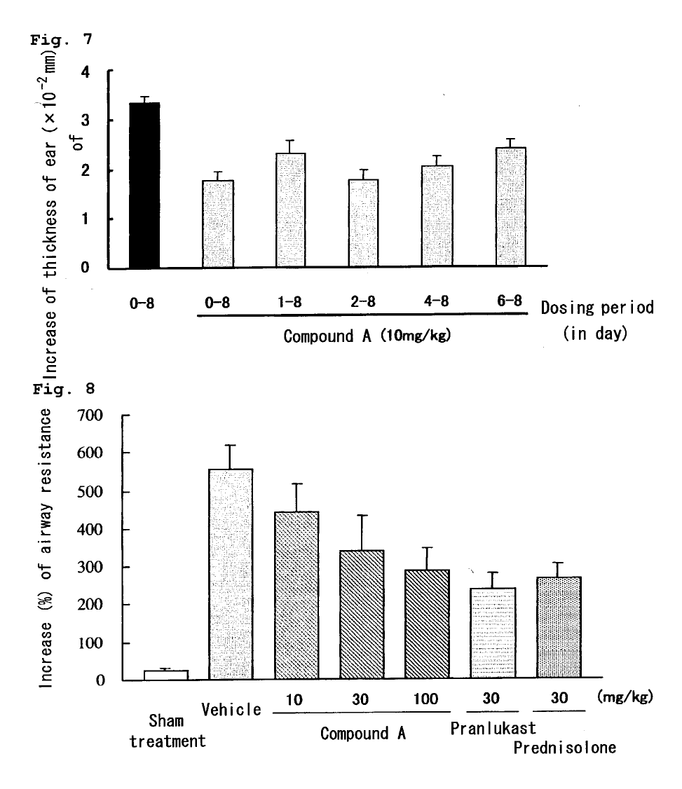

Before the antigen application and one hour, 24 hours and 8 days after the antigen application, the thickness of ear was measured with a dial thickness gauge (Yamazen Kikou). The difference between the value before the antigen application and the value at each measured time was used as a parameter of swelling indicator. FIGS. 4 through 6 show the results of each measurement. Furthermore, FIG. 7 shows the influence of the timing of the compound dosing on the swelling-suppressing effect 8 days after the antigen application.

* Results

The Compound A significantly suppressed ear swelling in any of the early phase (one hour after application), late phase (24 hours after application) and very late phase (8 days after application) in the IgE-dependent dermatitis model. Furthermore, the effect of the Compound A in the very late phase was observed in the case of the start of dosing later than the induction of the late phase.

2. Therapeutic Effect Using Asthma Model

Effect on antigen-induced early phase asthma, late phase asthma and airway hypersensitivity in guinea pig

*Preparation of Test Compound

A given amount of Compound A was suspended in aqueous 0.5% MC solution to 60 mg/mL. The test compound was further diluted to 20, 6, and 2 mg/mL on a needed basis. As positive controls, similarly, pranlukast hydrate (extracted from Onon under trade name (Ono Pharmaceutical Co., Ltd.)) of 6 mg/mL and prednisolone (Sigma) of 6 mg/mL were prepared.

* Active Sensitization and Antigen Challenge

Sensitization: Using an ultrasonic nebulizer (NE-U12; OMRON), a male Hartley guinea pig of age 6 weeks (Kudo, Co., Ltd.) was allowed to continuously inhale 1% OVA (ovalbumin; Sigma)—containing physiological saline for 10 minutes per day for consecutive 8 days.

Antigen challenge: One week after the last sensitization, the guinea pig was similarly allowed to inhale 2% OVA for 5 minutes. 24 hours before and one hour after the OVA challenge, metyrapone-containing physiological saline (Aldrich, 10 mg/mL) was dosed intravenously. 30 minutes before the OVA induction, pyrilamine-containing physiological saline (Sigma, 10 mg/kg) was dosed intraperitoneally.

* Dosing of Test Compound

For the 15-day period from the start of sensitization to the antigen challenge, the test compound was orally given at 5 mL/kg once daily. For 8 days for sensitization, the test compound was given one hour before sensitization. On the day of the antigen challenge, the test compound was given one hour before the challenge. As solvent controls, the vehicle was similarly dosed for OVA induction and physiological saline induction groups.

As positive controls, pranlukast hydrate was dosed one hour before the challenge, while prednisolone was dosed 16 hours and 2 hours before the challenge. Meanwhile, the animal was put at starved state from 16 hours to 18 hours before oral dosing.

* Measurement of Airway Resistance

Using total respiratory function analysis system (Pulmos-I, M.I.P.S. Company), the prep value was measured. Subsequently, specific airway resistance (referred to as sRaw hereinafter) per 100 breathes was measured individually one minute after OVA challenge and 2, 4, 5, 6, 7 and 8 hours thereafter and additionally once 22 to 26 hours thereafter. The average was designated sRaw of each measurement time. The increment ratio of sRaw was calculated by the following formula.

Increment ratio (%) of sRaw=[(sRaw of each measurement time−sRaw before challenge)/(sRaw before challenge)]×100

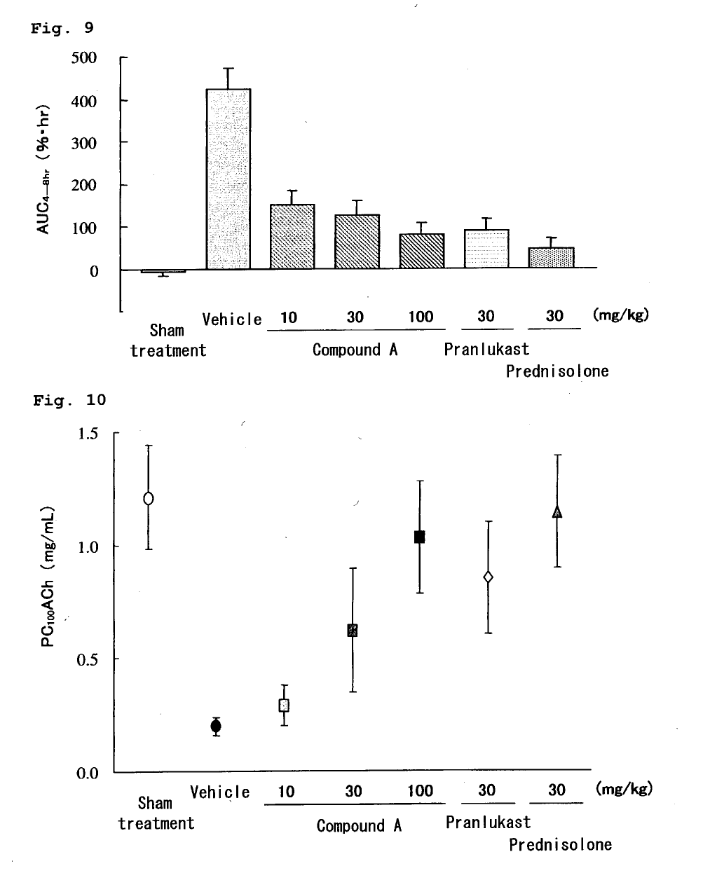

FIG. 8 shows the increment ratio of sRaw one minute after OVA challenge and FIG. 9 shows the increment ratio of sRaw (area under the curve: AUC_{4-8 hr}) over 4 to 8 hours after the challenge.

* Measurement of Airway Reactivity

22 to 26 hours after antigen challenge, physiological saline and acetylcholine (referred to as ACh hereinafter) solutions of 0.0625, 0.125, 0.25, 0.5, 1 and 2 mg/mL were sequentially inhaled individually for one minute, until the sRaw was 2-fold or more the baseline sraw (sRaw after inhalation of physiological saline). Based on the ACh concentration and the concentration-resistance curve of sRaw, the ACh concentration required for the sRaw to attain 100% increment from the baseline sRaw, namely PC100ACh was determined. The measurement results are shown in FIG. 10.

* Results

In the model, the Compound A suppressed any of antigen-induced early phase asthma response (sRaw immediately after antigen challenge), late phase asthma response (sRaw over 4 to 8 hours after antigen challenge) and airway hypersensitivity. The positive controls pranlukast hydrate and prednisolone also suppressed any of antigen-induced early phase asthma response, late phase asthma response and airway hypersensitivity.

3. Action on Leukotriene Generation

It is known that leukotriene (referred to as LTs hereinafter) is generated by basophils, mast cells and the like and is involved in the exacerbation of allergic disease, particularly allergic bronchial asthma.

1. Action on Leukotriene Generation from Human Basophils

* Preparation of Test Compound

A given amount of Compound A was prepared with DMSO (dimethyl sulfoxide) to 0.01 mM, which was then diluted with Tyrode solution (Sigma) to prepare 100 μM to 0.1 μM compound A solutions (1% DMSO solution). For cell action, the solutions were further diluted to 10 μM to 0.01 μM (0.1% DMSO solution).

* High Purification of Basophils

Using a syringe charged with 3.8% sodium citrate solution, 100 mL blood was obtained from human blood.

Using 10×HBSS (−) (10×Hank's balanced salt solution, GIBCO), Percoll (Amersham), and Milli Q water, Percoll-HBSS (−) layers of 1.070 g/mL, 1.079 g/mL, 1.088 g/mL were prepared to be overlaid, on which the blood was overlaid. Centrifuging the blood at 300×g, a cell fraction between the 1.070 g/mL Percoll-HBSS (−) layer and the 1.079 g/mL Percoll-HBSS (−) layer was recovered. A 3-fold volume of HBSS (−) was added to the recovered cell suspension, for centrifugation at 300×g at 4° C. for 7 minutes. After centrifugation, the supernatant was discarded, while the cells were rinsed once with HBSS (−). The cell population recovered thus was designated basophils.

* Preincubation

The basophils were prepared with Tyrode solution to 2.5×10⁶ cells/mL, to which 10 μg/mL recombinant human IL-3 (Genzyme/Techne) was added to a final concentration of 100 ng/mL. Immediately, the basophils were placed at 80 μL/well (2.5×10⁵ cells/well) on a round-bottom 96-well plate, for incubation in 5% CO₂ at 37° C. for 30 minutes.

* Addition of Test Compound

After preincubation, the test compound was added at 10 μL/well, for incubation in 5% CO₂ at 37° C. for 10 minutes. Tyrode solution containing 1% DMSO was added at 10 μL/well to the solvent control group.

* Addition of Anti-Human IgE Antibody

Anti-human IgE antibody diluted to 1, 3, 10, 30 and 100 μg/mL with Tyrode solution was added at 10 μL/well, for incubation in 5% CO₂ at 37° C. for 30 minutes (the final concentrations were individually 0.1, 0.3, 1, 3, and 10 μg/mL).

* LTs Assay

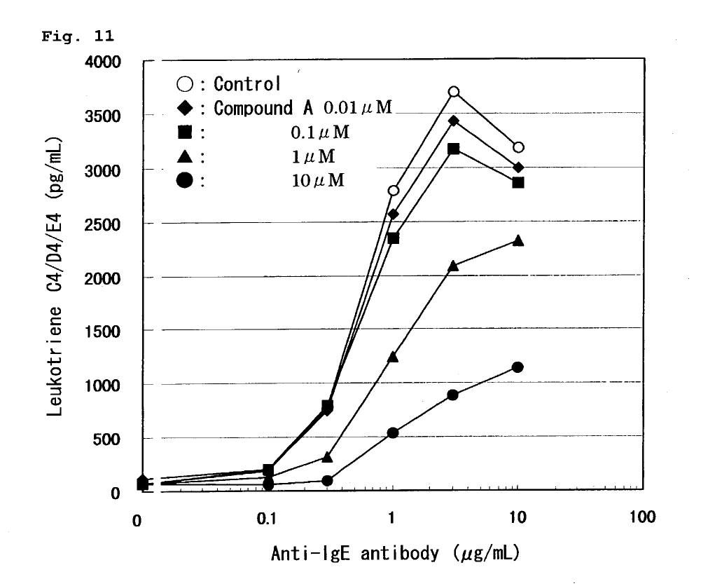

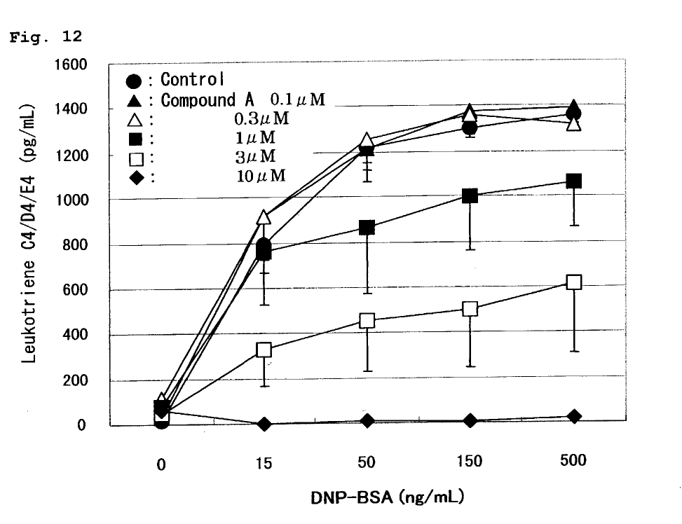

30 minutes after stimulation, the incubation mixtures were centrifuged at 3 000 rpm at 4° C. for 5 minutes, to recover the supernatants at 80 μL/well. The LTs amount in the supernatants was assayed according to the manufacturer's protocol of a LTs EIA kit (Amersham Pharmacia). The samples were diluted with Tyrode solution to 3-fold and 24-fold, for the assay. The assay results are shown in FIG. 11.

* Results

The Compound A exerted a suppressive action of the generation of leukotrienes (C4/D4/E4) from human basophils at the test.

2. Action on Leukotriene Generation from Rat Mast Cell Line

* Preparation of Test Compound

A given amount of Compound A was diluted and adjusted to 3, 1, 0.3, and 0.1 mM (100% DMSO solution)., Further, the resulting solutions were diluted with E-MEM culture medium (EAGLE-MEM; Nikken Bio-research Institute) to individually adjust the solutions to 100 to 1 μM (1% DMSO solution). For action on cell, the solutions were additionally diluted to 10 μM to 0.1 μM (0.1% DMSO solution).

* Preparation of PIPES Buffer

1 mM PIPES (Dojin Chemical Research Institute), 14 mM NaCl, 0.5 mM KCl, 0.06 mM MgCl₂, 0.1 mM CaCl₂, 0.55 mM glucose and 0.1% BSA (bovine serum albumin; Sigma) were prepared with purified water and were then adjusted to pH 7.4, using NaOH.

* Preparation of Anti-DNP IgE

1 mg/mL anti-DNP IgE (monoclonal murine anti-DNP IgE; Yamasa Corporation) was diluted to 1 000 fold with the PIPES buffer, to prepare 1 μg/mL solution.

* Preparation of DNP-BSA

10 mg/mL DNP-BSA was diluted to 10 μg/mL concentration with the PIPES buffer.

* Method for Culturing Rat Mast Cell Line

Culture medium: E-MEM culture medium containing heat-inactivated 10% FCS (fetal calf serum; Morgate Biotech), 100 units/mL penicillin, and 100 μg/mL streptomycin (in the form of penicillin/streptomycin; GIBCO).

* Cell Preparation

After a rat mast cell line RBL-2H3 (Human Science; 1×10⁶ cells/mL/tube) was centrifuged and rinsed with the culture medium, the cell line was resuspended in the culture medium, for culturing in a 75-cm² flask (Falcon 353136) for 3 days. After sub-culturing, additionally, the cell line was cultured in a 225-cm² flask (Corning 431082) for 2 days. It was confirmed that the cell line was at a semi-confluency state (at 60 to 70% confluency). Then, the cell line was rinsed in HBSS and detached with trypsin-EDTA. After the cells were recovered, the cells were centrifuged and rinsed in the culture medium, and were then resuspended in the culture medium. The cells were adjusted to 2×10⁵ cells/mL and were then placed at 250 μl/well on a 96-well flat bottom culture plate (Falcon 3072), for culturing in 5% CO₂ at 37° C. for 20 hours.

* Antigen Sensitization

Discarding the culture medium from the plate, the cells were rinsed in HBSS, followed by addition of 150 ng/mL anti-DNP IgE at 100 μL/well, for incubation at 37° C. for 30 minutes for cell sensitization.

* Addition of Test Compound

Discarding the culture medium from the plate, the cells were rinsed in HBSS, followed by addition of the culture medium at 80 μL/well and subsequent addition of the Compound A diluted with the culture medium to 1, 3, 10, 30, and 100 μM (the final concentrations were individually 0.1, 0.3, 1, 3, and 10 μM in the final DMSO concentration of 0.1%) at 10 μL/well, for incubation at 37° C. for 10 minutes.

* Antigen Stimulation

DNP-BSA diluted with the culture medium to 150, 500, 1500, and 5000 ng/mL was added at 10 μL/well (the final concentrations were individually 15, 50, 150 and 500 ng/mL), for incubation at 37° C. for 30 minutes.

* LTs Assay

30 minutes after the antigen stimulation, the supernatant was recovered at 20 mL/well, to assay the LTs amount by the manufacturer's protocol of a LTs EIA kit (Amersham Pharmacia). The assay results are shown in FIG. 12.

* Results

The Compound A exerted a suppressive action on the leukotriene generation (C4/D4/E4) from rat mast cell line at the test.

4. Cannabinoid Receptor-Binding Assay

The Compound A is known as a regulating substance selective for peripheral cell type cannabinoid receptor (IC₅₀ is 3436 nM to CB1 or is 0.087 nM to CB2) (Pharmacological test results, Table 33, Examples 3 to 5 in JP-A-2000-256323).

3. Actions of CB2 Inverse Agonist and CB2 Agonist on Murine IgE-Dependent Allergic Dermatitis Reactions

Using a triphase dermatitis model induced by antigen after a mouse was passively sensitized with IgE, the actions of CB2 inverse agonist and CB2 agonist were examined.

Test Method

* Animal: Female BALB/c mouse of age 8 to 10 weeks (SLC) was used.

* Preparation of Test Compound

Solvent preparation: MC was dissolved in distilled water, to prepare aqueous 0.5% MC solution.

Preparation of test compound: A given amount of Compound A was suspended in the solvent to prepare 0.01, 0.1 and 1 mg/mL suspensions.

A positive control prednisolone of 0.5 mg/mL was prepared as an MC suspension similarly as described above, while as comparative controls, a CB2 specific agonist HU-308 of 1 and 5 mg/mL and a CB2 specific inverse agonist SR144528 of 0.01, 0.1 and 1 mg/mL were also prepared as MC suspensions similarly.

* Passive Sensitization

Anti-DNP IgE (antibody against DNP; Yamasa Corporation) was prepared to 15 μg/mL with physiological saline. 0.2 mL of the resulting solution was given through caudal vein to the mouse.

* Antigen Preparation and Application

Antigen preparation: DNFB (2,4-dinitrofluorobenzene) was prepared with a mixed solution of acetone and olive oil (3:1, v/v) to 0.15% (w/v) on a needed basis.

Antigen application: 25 μl each of the antigen solutions was applied on the surface and back surface of both the ears, 24 hours after the dosing of the anti-DNP IgE.

* Dosing of Test Compound

From the next day of the antigen application to the day 8 after the antigen application, the test compound was orally dosed at 10 mL/kg once a day in total of 9 times. Herein, the test compound was dosed one hour before the antigen application on the day of antigen application while the test compound was dosed one hour before the measurement of the thickness of ear on the day 8 after the antigen application.

* Measurement of Auricle Swelling

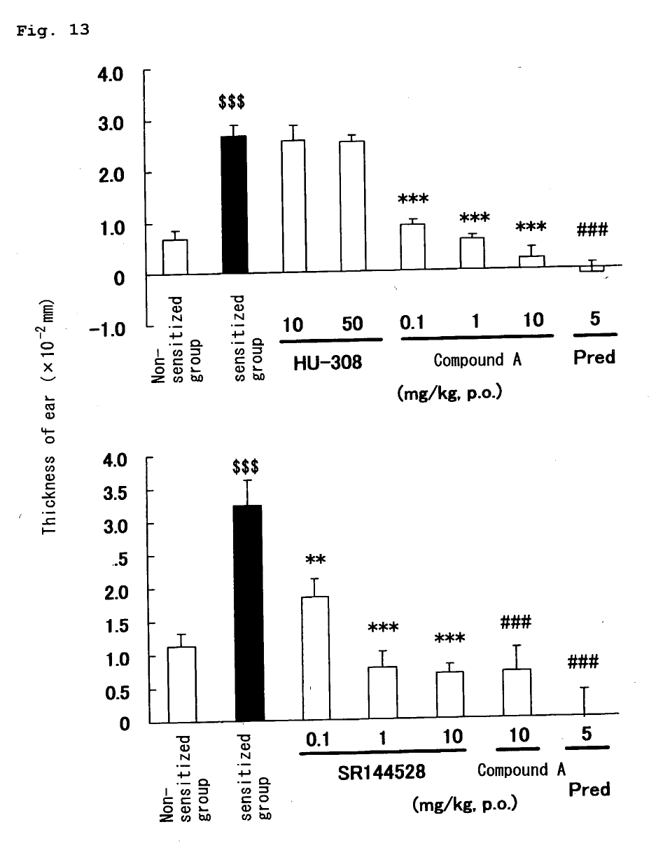

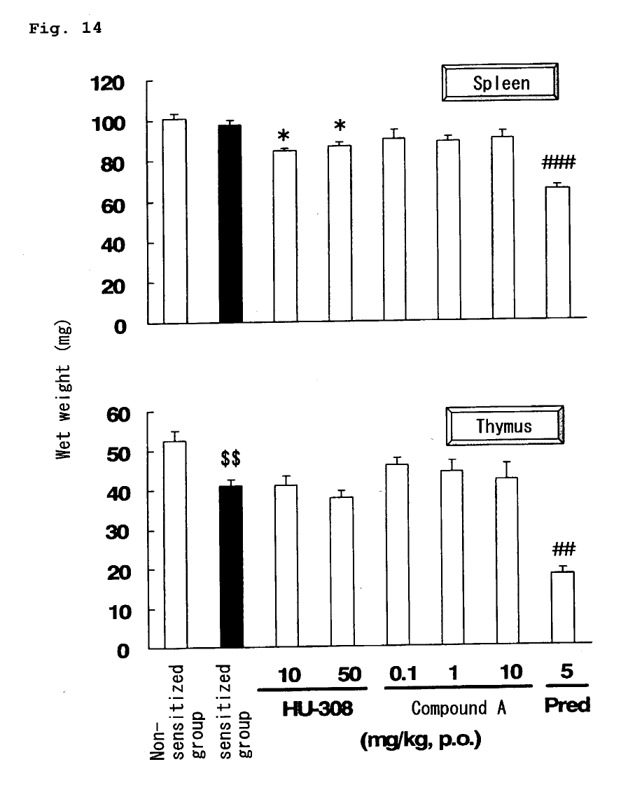

Before and 8 days after the antigen application, the thickness of ear was measured with a dial thickness gauge (Yamazen Kikou). The difference between the value before the antigen application and the value at each time period was used as a parameter of swelling. FIG. 13 shows the results of the measurement.

* Measurement of Organ Weight

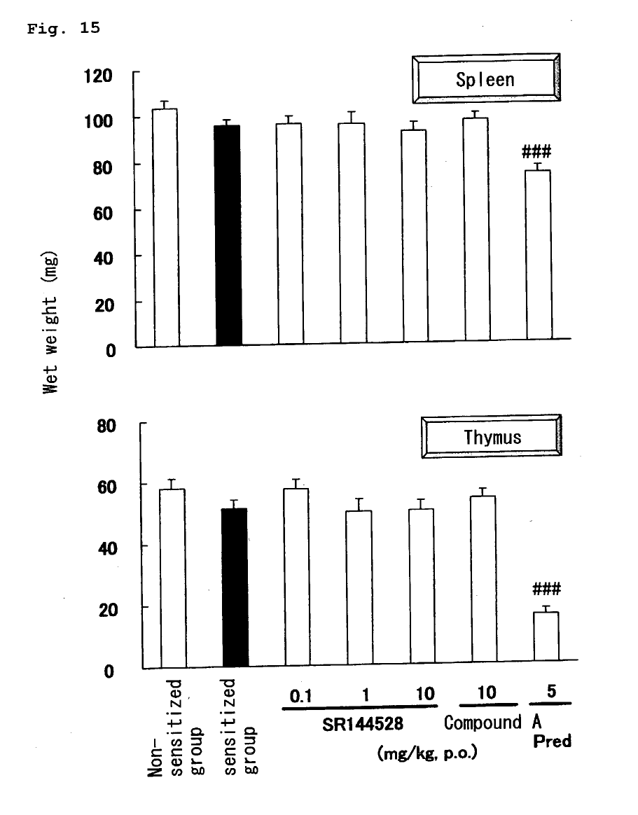

After the measurement of ear swelling, spleen and thymus were resected, to weigh the wet weights. The results of the measurement are shown in FIGS. 14 and 15.

* Results

Compound A significantly suppressed ear swelling at any dose of 0.1, 1 and 10 mg/kg at the very late phase (8 days after application). Further, SR144528 as an inverse agonist showed significant effect at 0.1 mg/kg or higher. On the other hand, HU-308 as a CB2 agonist did not show any pharmacological efficacy at any of 10 and 50 mg/kg. Spleen and thymus were weighed. The results are that the Compound A and SR144528 did not involve any apparent change in the weights, while prednisolone significantly suppressed the weights of both the organs. It was observed that the spleen weight in the animals dosed with HU-308 was significantly decreased.

4. Ear Swelling Induced by CB2 Agonist and Action of Compound A

Because CB2 inverse agonist showed efficacy in the IgE-dependent allergic dermatitis model, it was examined whether or not the stable substance of an endogenous ligand candidate 2-arachidonoylglycerol, namely 2-arachidonoylglycerol ether (2-AG-E) and a specific CB2 agonist HU-308 directly induced ear swelling. Additionally, comparison with ear swelling induced by arachidonic acid (AA) was done. Further, the action of Compound A on the influence of CB2 agonist on ear was examined.

Test Method

* Animal: Female BALB/c mouse (SLC) of age 8 to 10 weeks was used.

* Preparation and Application of Test Substances

Synthetically prepared 2-AG-E and HU-308 were individually prepared with acetone to 1, 10% (w/v) and 10% (w/v), respectively, while AA was prepared with acetone to 1.25% (w/v), on a needed basis. 10 μl each of these substances was coated on the surface and back face of left ear.

* Preparation of Solvent and Compound A

Solvent preparation: MC was dissolved in distilled water, to prepare aqueous 0.5% MC solution.

Preparation of Compound A: A given amount of Compound A was suspended in the solvent, to prepare 0.001, 0.01, 0.1 and 1 mg/mL suspensions.

* Dosing of Compound A

The solvent or the Compound A was orally given at 10 mL/kg. One hour later, 10 μl each of 10% (w/v) 2-AG-E was coated on the surface and back surface of left ear.

* Measurement of Ear Swelling

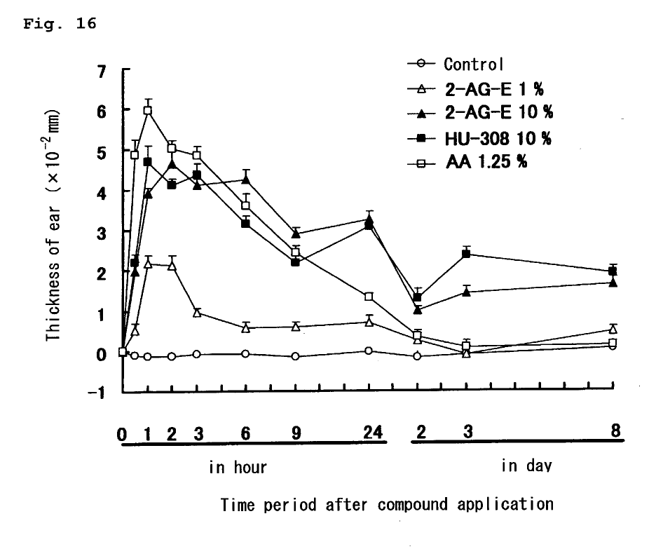

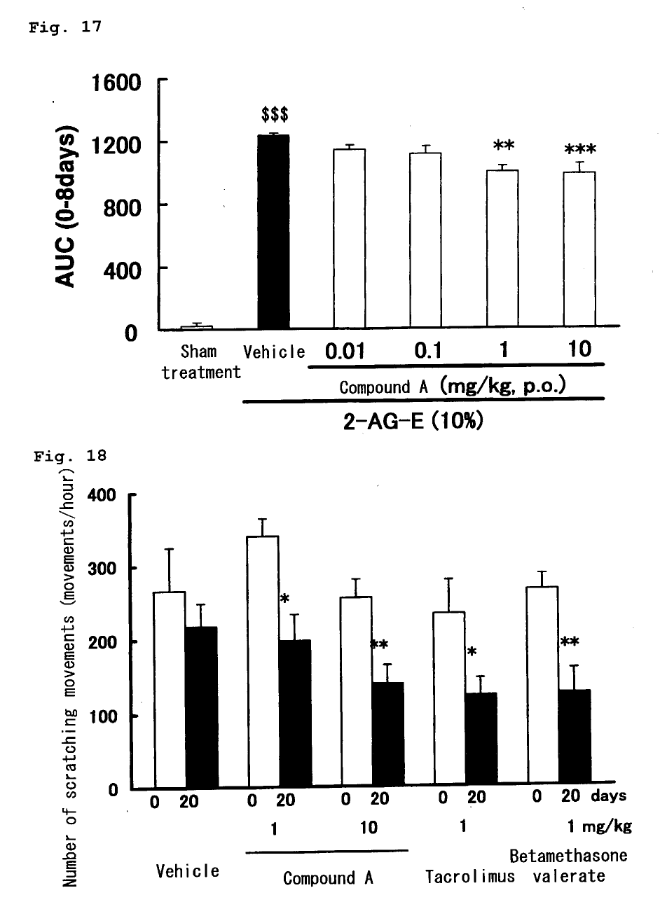

Before application of the test substance, and one, 2, 3, 6, 9 and 24 hours after the application and 2, 3 and 8 days thereafter, the thickness of ear was measured with a dial thickness gauge (Yamazen Kikou). The difference between the value before the antigen application and the value at each measured time was used as a parameter of swelling. For the evaluation of the Compound A, the area under the curve obtained from the change of ear swelling over time up to the day 8 after 2-AG-E application was calculated and used. The results of individual measurements are shown in FIGS. 16 and 17.

* Results

Via the application of 2-AG-E, ear swelling with a peak one hour to two hours later, depending on the 2-AG-E concentration was observed. Ear swelling was sustained at the 10% concentration up to the day 8 after application. HU-308 also induced sustainable ear swelling similarly. On the other hand, AA induced swelling with a peak one hour after the application and at almost the same level as that of 10% 2-AG-E. 2 days later, however, the ear swelling was back to the initial level.

The compound A suppressed ear swelling due to 10% 2-AG-E application in a manner dependent on the dose thereof and showed significant effect at the doses of 1 and 10 mg/kg.

5. Effect on Spontaneous Scratching Reaction of NC Mouse

Itching is one of main symptoms in the field of dermatology of atopic dermatitis, urticaria and contact dermatitis. However, most of the mechanism of the onset has not yet been elucidated. Thus, no pharmaceutical agent suppressing itching greatly and having less side effects has been developed.

Currently, NC mouse is used as an animal model of atopic dermatitis. No dermatitis or scratching action is observed when the mouse is kept in environment under control of atmospheric microorganisms (in SPF environment). However, scratching action together with the onset of dermatitis since about week 8 can be observed when the mouse is kept in conventional environment. It is known that the symptoms progress as chronic symptom (J. Dermatol. Sci., 25, 20-28, 2001).

Test Method

* Preparation of Test Compound

Solvent preparation: MC was dissolved in tap water, to prepare aqueous 0.5% (w/v) MC solution.

* Preparation of test compound: A given amount of Compound A was suspended in the solvent, to prepare 1 mg/mL and 0.1 mg/mL suspensions. As positive controls, similarly, positive controls betamethasone valerate (Sigma) and tacrolimus hydrate (extracted from Prograf (Fujisawa Pharmaceutical Co., Ltd.)) were prepared to 1 mg/mL. Betamethasone valerate is one of adrenocorticosteroids approved of the efficacy for the therapeutic treatment of atopic dermatitis, while tacrolimus hydrate is a therapeutic agent of atopic dermatitis, which is known as immunosuppressor as described above.

* Feeding of Animal and Screening Method

Male NC/Jic mice of age 4 weeks (CLEA JAPAN, INC.) were kept in the same cage as mice (A) infected with rodent mite (Myoba musculi) at the onset of severe dermal lesions, for 12 days. Thereafter, the mice (A) were taken out from the cage. The remaining mice of age 16 weeks were used.

* Feeding conditions: The mice were fed with a solid feed CA-1 (CLEA JAPAN INC.) ad libitum and with tap water as drinking water ad libitum, at a temperature of 22±2° C. and a humidity of 55±10% under lighting from 8:00 a.m. to 20:00 p.m.

From 10 days before the start of the experiment, the number of scratching behavior with murine hind legs was visually counted (for 20 minutes; once daily) over 2 days or 3 days. Among the plural mice counted, mice with 50 or more scratching movements on average per day were screened and used.

* Dosing of Test Compound

The test compound was orally dosed at 10 ml/kg once daily over 3 weeks.

* Test Method

The behavior of the mice was observed in unattended environment with a video camera, to count the scratching motion with hind legs for one hour. Generally, mouse shows several scratching motions for about one second. When a series of such motions was defined one scratching behavior, all such scratching movements were counted irrespective of scratched sites. The measurement was done on the day of the start of dosing, and one day, 3, 6, 10, 13, 17 and 20 days after the dosing. The results are shown together with the results of the positive controls in FIG. 18.

* Results

Compared with the control dosed with the solvent alone, the Compound A suppressed the number of scratching movement in the scratching reaction model. Further, the positive controls tacrolimus hydrate and betamethasone valerate also suppressed the number of scratching behavior.

The above results indicate that cannabinoid receptor-regulating substances, particularly peripheral cell type cannabinoid receptor (CB2)-selective inverse agonists such as Compound A and SR144528 are effective as therapeutic agents of allergic disease.

Particularly, the cannabinoid receptor-regulating substances were effective for the therapeutic treatment of asthma and atopic dermatitis occurring in a complex of allergic reactions of early phase, late phase and very late phase. Further, the effect on the suppression of allergic dermatitis in the late phase and very late phase is expected to be effective for chronic dermatitis. Thus, cannabinoid receptor-regulating substances, particularly CB2-selective inverse agonists such as Compound A and SR144528 are effective for intractable allergic dermatitis, particularly atopic dermatitis for which only steroids and immunosuppressor tacrolimus hydrate have prominent effects.

Further, the cannabinoid receptor-regulating substances, particularly the CB2-selective inverse agonists are effective as anti-asthma agents reducing any symptom of antigen-induced early phase asthma, late phase asthma and airway hypersensitivity of allergic asthma and may be effective for intractable asthma.

Still further, it was observed that cannabinoid receptor-regulating substances, particularly CB2-selective inverse agonists such as Compound A could reduce scratching movement possibly due to allergic reaction at the mouse scratching reaction test.

Additionally, the cannabinoid receptor-regulating substances, particularly the CB2-selective inverse agonists are potential pharmaceutical agents with safety profiles with no systemic immunosuppression, which suggests possible applicability as oral agent.

Compound A and SR144528 are known to have strong selective action on cannabinoid receptors, particularly CB2 receptor. The test results that CB2-selective agonist HU-308 and a derivative of a cannabinoid endogenous ligand 2-AG, namely 2-AG-E did not show anti-allergic action and that 2-AG-E induced allergic response while the Compound A suppressed even the allergic response, support that CB2-selective inverse agonists are useful as anti-allergic agents.

Thus, the effects of Compound A and SR144528 on the therapeutic treatment of allergic disease may be ascribed to the action of cannabinoid receptors. Particularly, the Compound A and SR144528 may be effective as pharmaceutical agents with different action mechanisms from those of the existing therapeutic agents of allergic disease, for example for symptoms with resistance against the existing pharmaceutical agents. Furthermore, it was also observed that the action of Compound A on leukotriene inhibition might enhance the therapeutic effect thereof.

Compound A and SR144528 have characteristic chemical structures different from each other but have a common feature that they are CB2-selective inverse agonists in view of pharmacological action. These findings suggest that the CB2-selective inverse agonists are effective as a therapeutic agent of allergic disease.

Cannabinoid receptor-regulating substances are effective as therapeutic agents of allergic disease such as asthma and atopic dermatitis. Particularly, regulating substances selectively acting on peripheral cell type cannabinoid receptor, more particularly regulating substances acting as inverse agonist are effective for chronic and intractable allergies diseases, for which existing therapeutic agents of allergic disease have low effects, and are potentially safe pharmaceutical agents.

What is claimed is:

1. A therapeutic agent of allergic disease, which contains as the active ingredient a cannabinoid receptor-regulating substance.

2. A therapeutic agent of allergic disease according to claim 1, where the cannabinoid receptor-regulating substance is a regulating substance selective for the peripheral cell type cannabinoid receptor.

3. A therapeutic agent of allergic disease according to claim 1 or 2, where the cannabinoid receptor-regulating substance is a 2-oxoquinoline compound represented by the following general formula [I] or a pharmaceutically acceptable salt thereof: 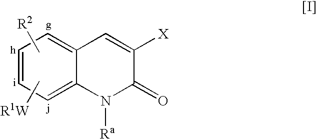
[in the formula,
W represents —O—, —S(O)ₜ—, —CR³R⁴—, —NR⁵—, —NR⁵CO—, —CONR⁵—, —COO— or —OCO— (in the formulas, R³ and R⁴ may be the same or different and independently represent hydrogen atom or alkyl; R⁵ represents hydrogen atom or alkyl; and t represents 0 or an integer of 1 and 2);
R¹ represents hydrogen atom, alkyl, alkenyl, alkynyl, aryl, arylalkyl, heteroaryl, heteroarylalkyl, cycloalkyl or cycloalkylalkyl; the individual groups in R¹ except for hydrogen atom may or may not be substituted with alkylamino, amino, hydroxyl group, alkoxy, carboxyl, alkoxycarbonyl, acyl, acyloxy, acylthio, mercapto, alkylthio, alkylsulfinyl or alkylsulfonyl; the individual groups except for hydrogen atom and alkyl may or may not be substituted with alkyl;
R² represents hydrogen atom, alkyl, —OR⁶ (in the formula, R⁶ represents hydrogen atom, alkyl, alkenyl, alkynyl, aryl, arylalkyl, heteroaryl, heteroarylalkyl, cycloalkyl or cycloalkylalkyl), —NR⁷R⁸ (in the formula, R⁷ and R⁸ may be the same or different and individually represent hydrogen atom, alkyl, alkenyl, alkynyl, acyl, aryl, arylalkyl, heteroaryl, heteroarylalkyl, cycloalkyl or cycloalkylalkyl; otherwise, R⁷ and R⁸ together with the adjacent nitrogen atom may form heteroaryl), or —(CH₂)_{u′}—S(O)ᵤR⁹ (in the formula, R⁹ represents hydrogen atom, alkyl, alkenyl or alkynyl; u and u′ independently represent 0 or an integer of 1 and 2),
where the individual groups in R² except for hydrogen atom may or may not be substituted with alkylamino, amino, hydroxyl group, alkoxy, alkoxycarbonyl, acyl, acyloxy, acylthio, mercapto, alkylthio, alkylsulfinyl or alkylsulfonyl; the individual groups except for hydrogen atom and alkyl may or may not be substituted with alkyl;
R^{a} represents hydrogen atom or alkyl;
X represents —COOR^{b}, —CONH₂, —CONR^{c}-(Alk^{a})ᵣ-R, —(CH₂)ₚ—OC(═Y)—NR^{d}-(Alk^{b})ₛ-R, —(CH₂)_{q}—NR^{e}—C(═Z)—(NR^{f})_{w}-(Alk^{c})ᵥ-R, —(CH₂)ₚ—OH or —(CH₂)_{q}—NR^{e}R^{e′}
{in the formula, R^{b}, R^{c}, R^{d} and R^{f} independently represent hydrogen atom or alkyl; R^{e} and R^{e′} independently represent hydrogen atom or alkyl; or R^{e} and R^{e′} together with the adjacent nitrogen atom may form heteroaryl;
Alk^{a}, Alk^{b} and Alk^{c} independently represent alkylene or alkenylene; the alkylene and alkenylene individualy may or not be substituted with represents hydroxyl group, carboxyl, alkoxycarbonyl, alkyl (the alkyl may or may not be substituted with hydroxyl group, alkoxy or alkylthio) or —CONR¹⁰R¹¹ (in the formula, R¹⁰ and R¹¹ may be the same or different and independently represent hydrogen atom or alkyl; or R¹⁰ and R¹¹ together with the adjacent nitrogen atom may form heteroaryl);
R represents aryl, heteroaryl, cycloalkyl, benzene-fused cycloalkyl or 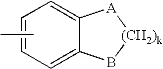
(in the formula, A and B independently represent oxygen atom, nitrogen atom or sulfur atom; and k represents an integer of 1 to 3), where the aryl and the heteroaryl individually may or may not be substituted with alkyl which may or may not be substituted with hydroxyl group, hydroxyl group, alkoxy, alkenyloxy, acyl, acyloxy, halogen atom, nitro, amino, sulfonamide, alkylamino, aralkyloxy, pyridyl, piperizino, carboxyl, alkoxycarbonyl, acylamino, aminocarbonyl, cyano or glucuronate residue; the cycloalkyl may or may not be substituted with hydroxyl group, alkoxy or ═O; the benzene-fused cycloalkyl may or may not be substituted with hydroxyl group or alkoxy;
r, s, v and w independently represent 0 or 1; Y and Z independently represent nitrogen atom, oxygen atom or sulfur atom; and p and q independently represent an integer of 1 to 4}].

4. A therapeutic agent of allergic disease according to claim 3, where the cannabinoid receptor-regulating substance is N-(benzo[1,3]dioxol-5-ylmethyl)-7-methoxy-2-oxo-8-pentyloxy-1,2-dihydroquinoline-3-carboxamide or a pharmaceutically acceptable salt thereof.

5. A therapeutic agent of allergic disease according to claim 1 or 2, where the cannabinoid receptor-regulating substance is selected from the group consisting of SR144528, HU-308, L-759 633, L-759 656, L-768 242, PRS-211 096, PRS-211 335, PRS-211 359, AM603, AM1703, AM1710 and AM1221.

6. A therapeutic agent of allergic disease according to claim 5, where the cannabinoid receptor-regulating substance is SR144528.

7. A therapeutic agent of allergic disease according to claims 1 through 6, where the cannabinoid receptor-regulating substance is an inverse agonist.

8. A therapeutic agent of allergic disease according to claims 1 through 7, where the allergic disease is allergic dermatitis.

9. A therapeutic agent of allergic disease according to claims 1 through 8, where the allergic disease is atopic dermatitis.

10. A therapeutic agent of allergic disease according to claims 1 through 7, where the allergic disease is allergic asthma.

11. A therapeutic agent of allergic disease according to claims 1 through 7 or claim 10, where the allergic disease is early phase asthma response or/and late phase asthma response or/and airway hypersensitivity.

12. A therapeutic agent of allergic disease according to claims 1 through 7, where the allergic disease is allergic rhinitis or allergic conjunctivitis.

13. A therapeutic agent of allergic disease according to claims 1 through 12, where the cannabinoid receptor-regulating substance concurrently has an action inhibiting leukotriene.

14. A pharmaceutical agent of a cannabinoid receptor-regulating substance for the therapeutic treatment of allergic disease due to cannabinoid agonist.

15. A therapeutic agent of allergic disease, the therapeutic agent containing as the active ingredient N-(benzo[1,3]dioxol-5-ylmethyl)-7-methoxy-2-oxo-8-pentyloxy-1,2-dihydroquinoline-3-carboxamide or a pharmaceutically acceptable salt thereof.

16. A therapeutic agent of atopic dermatitis, the therapeutic agent containing as the active ingredient N-(benzo[1,3]dioxol-5-ylmethyl)-7-methoxy-2-oxo-8-pentyloxy-1,2-dihydroquinoline-3-carboxamide or a pharmaceutically acceptable salt thereof.

17. A therapeutic agent of asthma, the therapeutic agent containing as the active ingredient N-(benzo[1,3]dioxol-5-ylmethyl)-7-methoxy-2-oxo-8-pentyloxy-1,2-dihydroquinoline-3-carboxamide or a pharmaceutically acceptable salt thereof.
